(19) United States
(12) Patent Application Publication
(10) Pub. No.: US 2017/0047525 A1
(43) Pub. Date: Feb. 16, 2017

(54) BENZOTRIIMIDAZOLE MATERIALS

(71) Applicant: THE US GOVERNMENT AS REPRESENTED BY THE SECRETARY OF THE ARMY-U.S. ARMY RESEARCH LABORATORY, Adelphi, MD (US)

(72) Inventors: Jianmin Shi, Rockville, MD (US); JOHN MATTHEW CHUDOMEL, NAPERVILLE, IL (US); ERIC WILLIAM FORSYTHE, ROCKVILLE, MD (US)

(73) Assignee: THE UNITED STATES GOVERNMENT AS REPRESENTED BY THE SECRETARY OF THE ARM Y, WASHINGTON, DC (US)

(21) Appl. No.: 14/822,918

(22) Filed: Aug. 11, 2015

(51) Int. Cl. H01L 51/00; C09K 11/02; C07D 487/14

(52) U.S. Cl. CPC H01L 51/0072; C07D 487/14; C09K 11/025; H01L 51/0065; H01L 51/5016

(57) ABSTRACT

Provided herein is an aromatic benzotriimizole compound of Formula I:

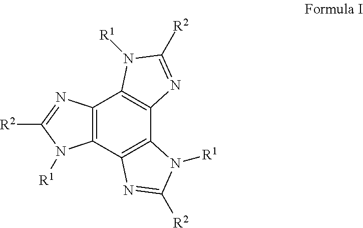

wherein,
R¹ is independently H, alkyl or aryl, wherein alkyl and the substitutions of aryl can include alkyl substituted with a reactive moiety; and
R² is independently alkyl, aryl, chloro, fluoro, bromo or cyano.

### GOVERNMENTAL INTEREST

The invention described herein may be manufactured, used and licensed by or for the U.S. Government.

### FIELD OF USE

This present invention relates to a new class of aromatic benzoimidizole organic materials, aromatic benzotriimidizole derivatives, methods for their preparation and organic electroluminescent devices and organic light emitting diode displays utilizing these compounds.

Imidazoles are a class of aromatic heterocycles, derivatives of imidizole. The amino acid histidine, and the related hormone histamine are members of the class. Many drugs contain an imidazole ring, such as antifungal drugs, nitroimidazole, and the sedative imidazole.

Imidazole is a highly polar compound and classified as aromatic due to the presence of a appropriate π-electrons. The proton of imidazole (un-ionized form) can be located on either of their two nitrogen atoms that are consisting of a pair of electrons from the protonated nitrogen atom and one from each of the remaining four atoms of the ring. This structural feature of imidazole is very important in search and design for new organic semiconductors. In 1997, Shi disclosed several classes of novel blue emitting materials belonging to the benzoimidizole class suitable for use as the emissive as well as the electron-transport materials in organic electroluminescent (EL) devices. [Device Research Conference Digest, 1997. 5th, P154, 1997], [U.S. Pat. No. 5,645,948], [U.S. Pat. No. 5,766,779]. This class of benzoimidizoles has been widely used in organic light emitting diode (OLED) display and organic solid state lighting technology field. These compounds include:

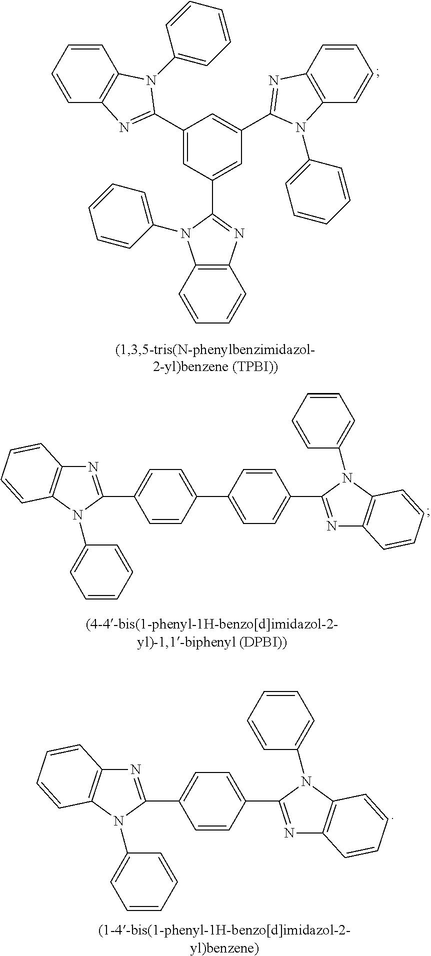 [SMILES: c1ccc(-n2c(-c3cc(-c4nc5ccccc5n4-c4ccccc4)cc(-c4nc5ccccc5n4-c4ccccc4)c3)nc3ccccc32)cc1 ; c1ccc(-n2c(-c3ccc(-c4ccc(-c5nc6ccccc6n5-c5ccccc5)cc4)cc3)nc3ccccc32)cc1 ; c1ccc(-n2c(-c3ccc(-c4nc5ccccc5n4-c4ccccc4)cc3)nc3ccccc32)cc1]

Among of these benzoimidazoles, TPBI was found particularly useful, and has been widely used. This success may be due to an unique aromatic polycyclic core and ring structure features that lead to its high thermal stability, chemical stability and high resistance to oxidation.

Up to now no reports can be found on how to obtain aromatic benzoimidizole derivatives. With more rigid aromatic polycyclic core and its highly electron-deficient aromatic ring system, the benzoimidizole might be expected to lead to formation of conductive and luminescent organic semiconductors. With such a system it might be possible to the increase in the core-core attractive interactions which might by design be configured to encourage or discourage molecular stacking.

Described herein are such derivatives, and a method of synthesizing them.

### SUMMARY

Provided herein is an aromatic benzotriimizole compound of Formula I:

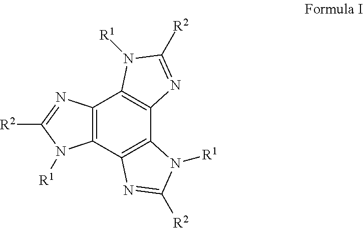

wherein,

R¹ is independently H, alkyl or aryl, wherein alkyl and the substitutions of aryl can include alkyl substituted with a reactive moiety (such as without limitation

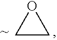

˜OH, ˜OCOCH═CH₂ [wherein “˜” represents the bond substituted for H]); and

R² is independently alkyl, aryl, chloro, fluoro, bromo or cyano.

Also provided, among other things, are electroluminescent devices wherein light emitting compounds comprise one or more compounds of the invention. Further provided are photovoltaic devices wherein light absorbing pigment comprises one or more compounds of the invention.

### DESCRIPTION OF THE DRAWINGS

So that the manner in which the above recited features of the present invention can be understood in detail, a more particular description of the invention, briefly summarized above, may be had by reference to embodiments, some of which are illustrated in the appended drawings. It is to be noted, however, that the appended drawings illustrate only illustrative embodiments of this invention and are therefore not to be considered limiting of its scope, for the invention may admit to other equally effective embodiments.

FIG. 1 shows absorption and emission spectra of 1,2,4,5,7,8-hexaphenyl-4,7-dihydro-1H-benzo[1,2-d:3,4-d′:5,6-d″]triimidazole (in dichloroethane) (Compound GIII-01);

FIG. 2, shows absorption and emission spectra of 2,5,8-tris(4-methoxyphenyl)-1,4,7-triphenyl-4,7-dihydro-1H-benzo[1,2-d:3,4-d′:5,6-d″]triimidazole (in dichloroethane) (Compound GIII-05);

FIG. 3, shows absorption and emission spectra of 2,5,8-tris(4-cyanophenyl)-1,4,7-triphenyl-4,7-dihydro-1H-benzo[1,2-d:3,4-d′:5,6-d″]triimidazole (in dichloroethane) (Compound GIII-02);

FIG. 4, shows absorption and emission spectra of 2,5,8-tris(4-(tert-butyl)phenyl)-1,4,7-triphenyl-4,7-dihydro-1H-benzo[1,2-d:3,4-d′:5,6-d″]triimidazole (in dichloroethane) (Compound GIII-04);

FIG. 5, shows absorption and emission spectra of 2,5,8-tris(3-fluoro-4-cyanophenyl)-1,4,7-triphenyl-4,7-dihydro-1H-benzo[1,2-d:3,4-d′:5,6-d″]triimidazole (in dichloroethane) (Compound GIII-08);

FIG. 6 shows the efficacy (cd/A as a function of current density) produced with a device incorporating compound GIII-02;

FIG. 7 shows the emission spectrum from Example 6; and

FIG. 8 shows an illustrative EL comprising compound of the invention as light emitting material.

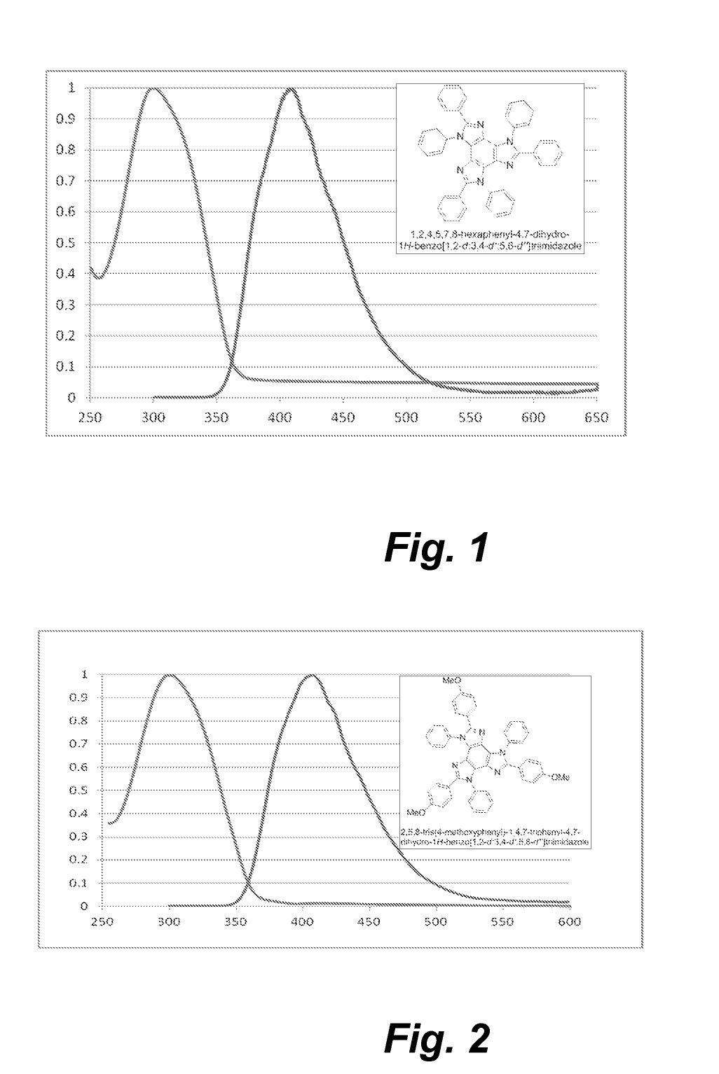

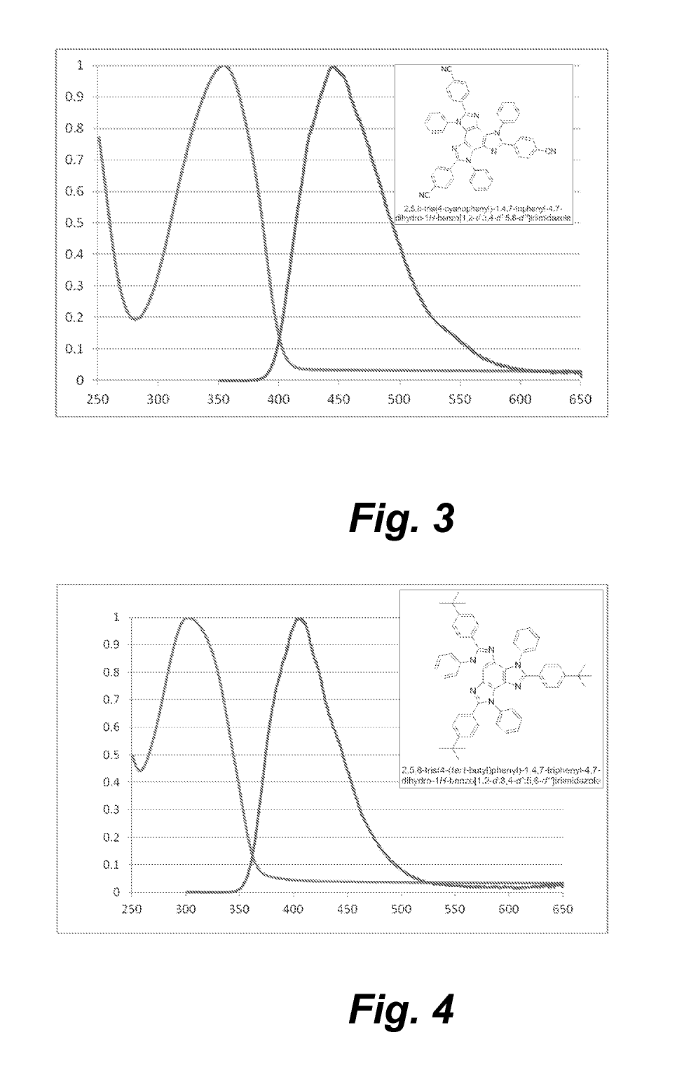

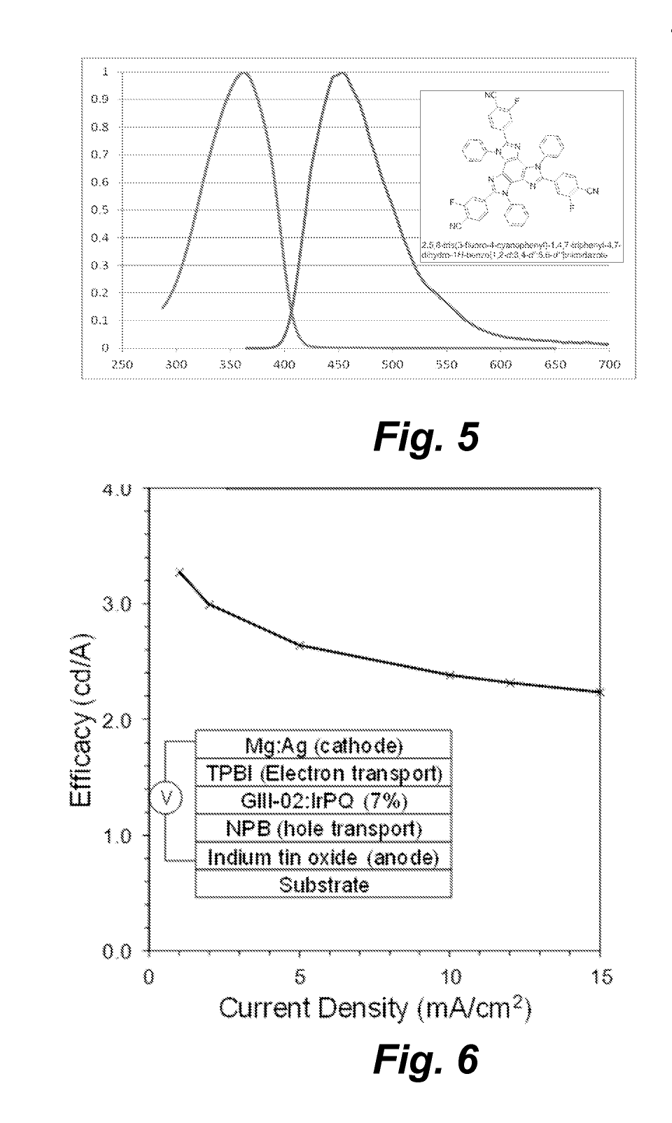

To facilitate understanding, identical reference numerals have been used, where possible, to designate comparable elements that are common to the figures. The figures are not drawn to scale and may be simplified for clarity. It is contemplated that elements and features of one embodiment may be beneficially incorporated in other embodiments without further recitation.

### DETAILED DESCRIPTION

To obtain the present invented benzotriimidazole class of materials can be carried out in two stages. The first stage is to obtained the intermediate N1,N3,N5-triphenylbenzene-1,2,3,4,5,6-hexaamines. In an exemplary process, one can start with for example 1,3,5-tribromobenzene, which was easily nitrated by a two step process to produce 1,3,5-tribromo-2,4,6-trinitrobenene in very high yield. This 1,3,5-tribromo-2,4,6-trinitrobenene is reacted with phenylamine(s) to give 2,4,6-trinitro-N1,N3,N5-triphenylbenzene-1,3,5-triamine. The intermediate N1,N3,N5-triphenylbenzene-1,2,3,4,5,6-hexaamine is obtained followed by reduction. The second stage is to produce compounds of the benzotriimidazole class. Intermediate N1,N3,N5-triphenylbenzene-1,2,3,4,5,6-hexaamine is reacted with aryl chloride acids using for example N-methyl-2-pyrrolidone (NMP) as solvent to produce N,N′,N″-(2,4,6-tris(phenylamino)benzene-1,3,5-triyl)tribenzamide in high yield. A compound of the new benzotriimidazole class of materials is obtained by acid catalyzed condensation using phosphoryl chloride (POCl3) as solvent.

In more generalized terms, the first stage reactions are as follows:

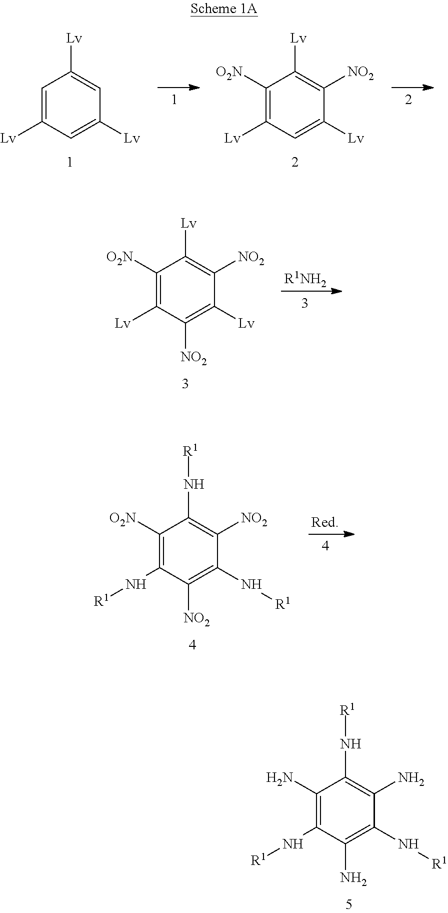

In the foregoing Scheme 1A, Lv is an appropriate leaving group, as will be recognized by those of skill in the art. In reaction 1, compound 1 is nitrated. In reaction 2, compound 2 is further nitrated with fuming HNO₃/H₂SO₄. In reaction 3, Lv is substituted with R¹NH₂. In reaction 4, the nitro groups are reduced to amines, such as with appropriate tin (Sn) reflux.

The second stage is as follows:

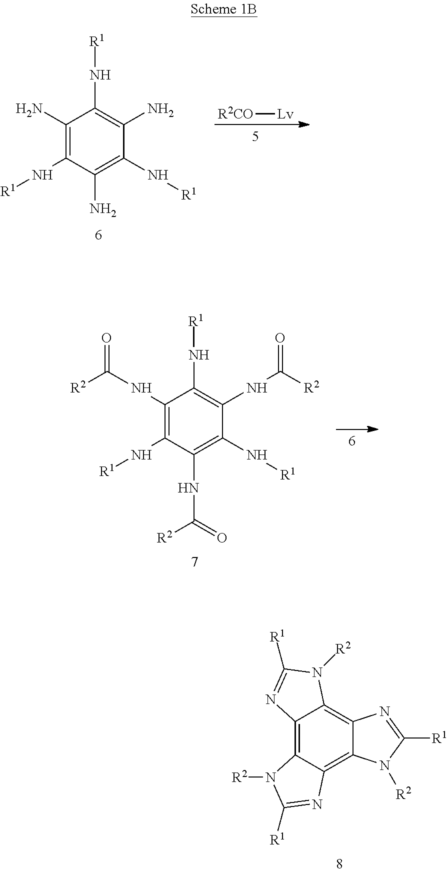

In the above Scheme 1B, Lv is independently an appropriate leaving group. In reaction 6, ring closure can be for example by acid catalyzed condensation, such as acid catalyzed condensation using phosphoryl chloride (POCl₃) as solvent.

The first part of the scheme is exemplified by the following:

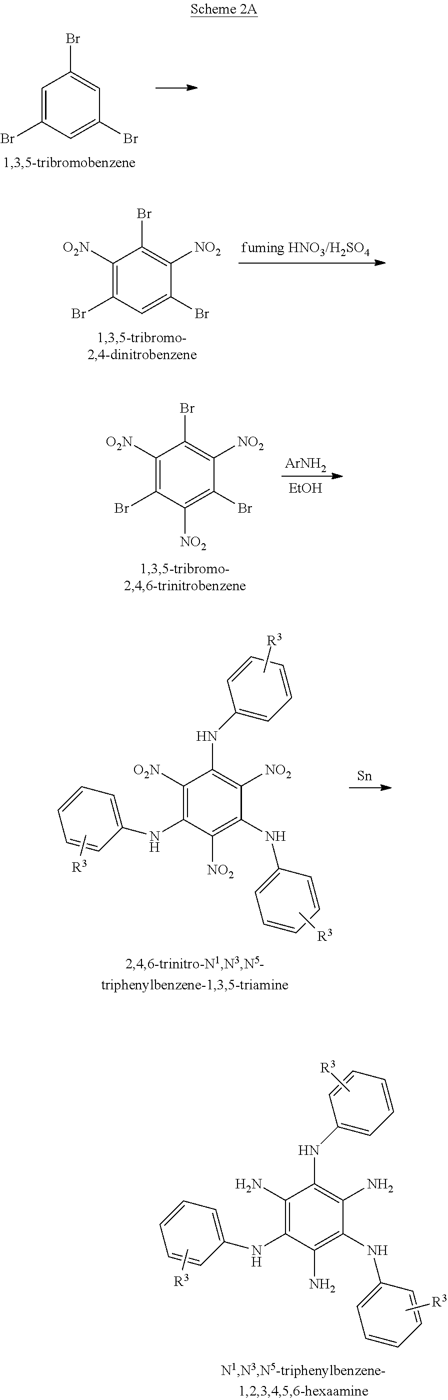

The second stage of the reaction scheme can be exemplified with the following:

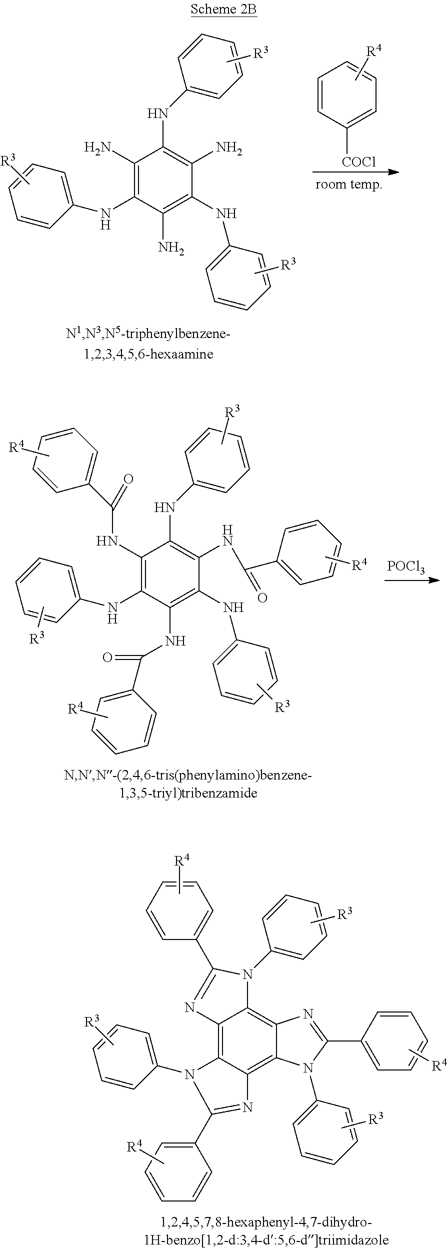

As used in the claims hereof, “alkyl” can be of 1 to 24 carbon atoms (straight or branched). In embodiments, alkyl is 1 to 10. Alkyl can be substituted (such that there are up to 24 carbon atoms). Nonlimiting substitutions include hydroxy, halo, alkyl, haloalkyl, alkenyl, haloalkenyl, alkynyl, haloalkynyl, alkoxy, haloalkoxy, alkylthio, haloalkylthio, arylthio, —CONH(Alkly), —O-Alkylene-O-Alkyl, —CO(Alkly), —CO—O-Alkly, and the like.

As used in the claims hereof, “aryl” is an aromatic ring system with 5 to 48 carbon atoms, including substitutions. The ring atoms can include heteroatoms. Nonlimiting exemplary aryls include for example phenyl and naphthyl, furyl, thienyl, pyridyl and quinolinyl. Nonlimiting substitutions include hydroxy, halo, alkyl, haloalkyl, alkenyl, haloalkenyl, alkynyl, haloalkynyl, alkoxy, haloalkoxy, alkylthio, haloalkylthio, arylthio, nitro, —CONH(Alkly), —O-Alkylene-O-Alkyl, —CO(Alkly), —CO—O-Alkly, and the like.

In embodiments, the reactive moieties are composed of carbon, hydrogen and oxygen. In embodiments, the reactive moieties are attached at or near the terminal of an alkyl group, such that crosslinking reaction with external molecules or polymerization is facilitated.

In a Group I subset of compounds, R² is aryl. Nonlimiting examples of Group I compounds include, for example:

TABLE A
(General sub-formula found above each R¹ designation)

| | |
|---|---|
| Compound | |
| Designation | R¹ |

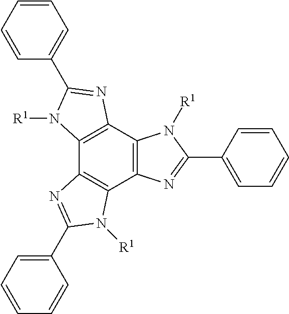

| | |
|---|---|
| GI-01 | H |
| GI-02 | 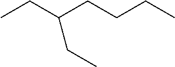 |
| GI-03 | 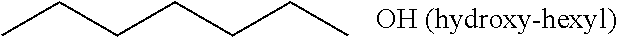 |
| GI-04 | 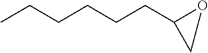 |
| GI-05 | 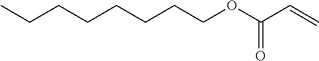 |

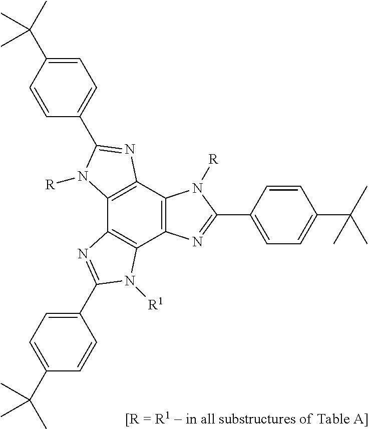

| | |
|---|---|
| GI-06 | H |
| GI-07 | 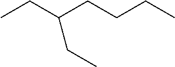 |
| GI-08 | 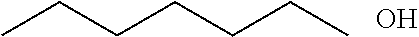 |
| GI-09 | 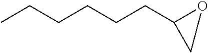 |
| GI-10 | 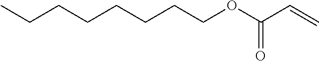 |

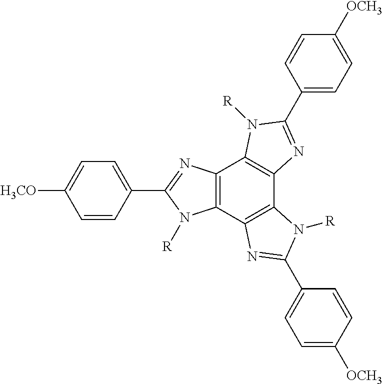

| | |
|---|---|
| GI-11 | H |
| GI-12 | 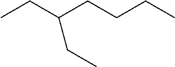 |
| GI-13 | 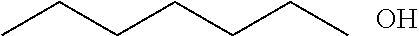 |
| GI-14 | 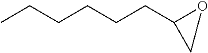 |
| GI-15 | 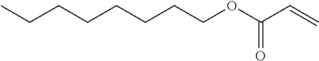 |

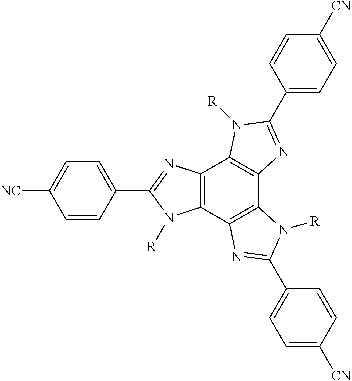

| | |
|---|---|
| GI-16 | H |
| GI-17 | 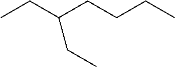 |
| GI-18 | 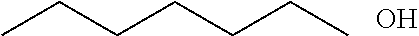 |
| GI-19 | 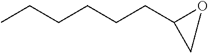 |
| GI-20 | 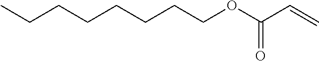 |

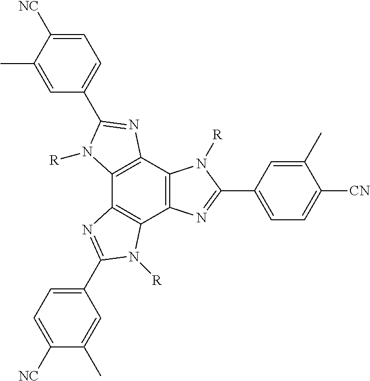

| | |
|---|---|
| GI-21 | H |
| GI-22 | 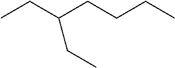 |
| GI-23 | 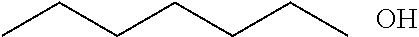 |
| GI-24 | 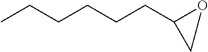 |
| GI-25 | 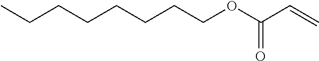 |

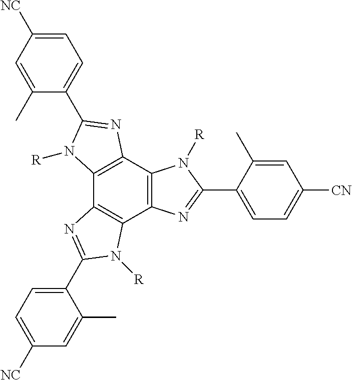

| | |
|---|---|
| GI-26 | H |
| GI-27 | 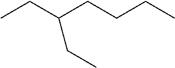 |
| GI-28 | 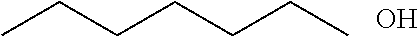 |
| GI-29 | 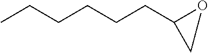 |
| GI-30 | 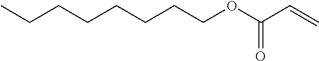 |

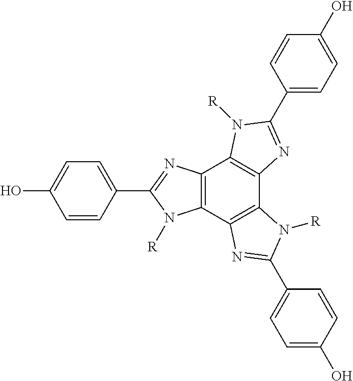

| | |
|---|---|
| GI-31 | H |
| GI-32 | 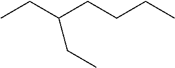 |
| GI-33 | 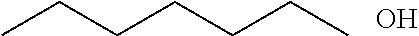 |
| GI-34 | 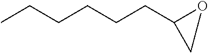 |
| GI-35 | 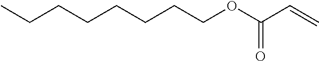 |

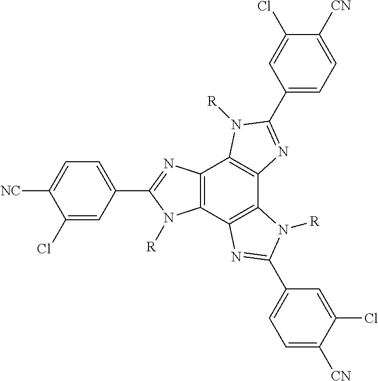

| | |
|---|---|
| GI-36 | H |
| GI-37 | 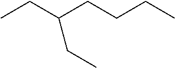 |
| GI-38 | 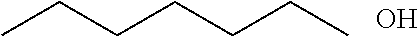 |
| GI-39 | 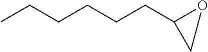 |
| GI-40 | 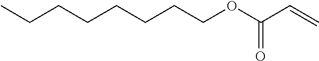 |

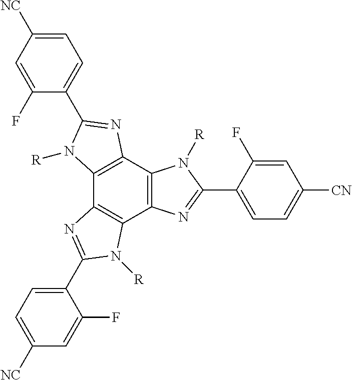

| | |
|---|---|
| GI-41 | H |
| GI-42 | 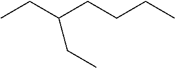 |
| GI-43 | 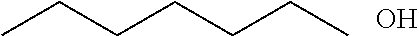 |
| GI-44 | 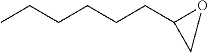 |
| GI-45 | 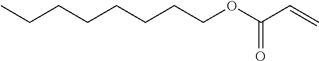 |

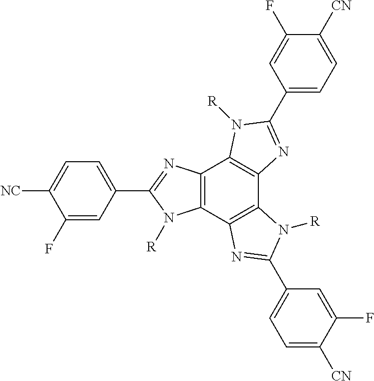

| | |
|---|---|
| GI-46 | H |
| GI-47 | 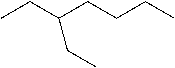 |
| GI-48 | 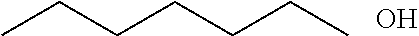 |
| GI-49 | 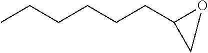 |
| GI-50 | 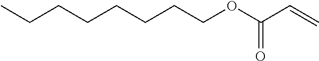 |

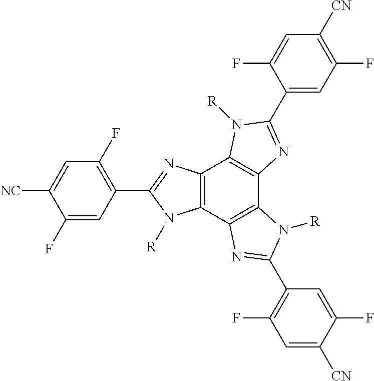

| | |
|---|---|
| GI-51 | H |
| GI-52 | 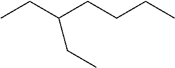 |
| GI-53 | 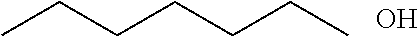 |
| GI-54 | 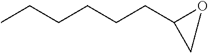 |
| GI-55 | 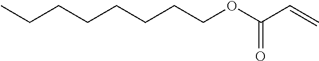 |

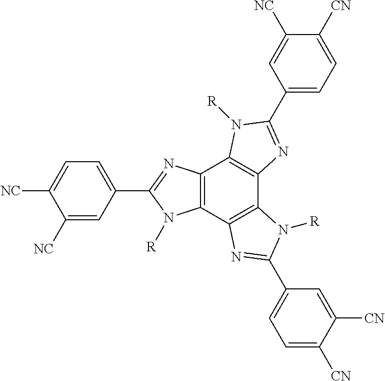

| | |
|---|---|
| GI-56 | H |
| GI-57 | 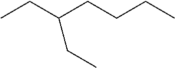 |
| GI-58 | 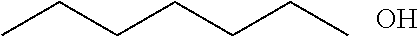 |
| GI-59 | 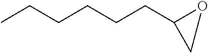 |
| GI-60 | 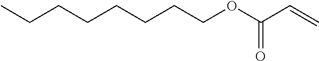 |

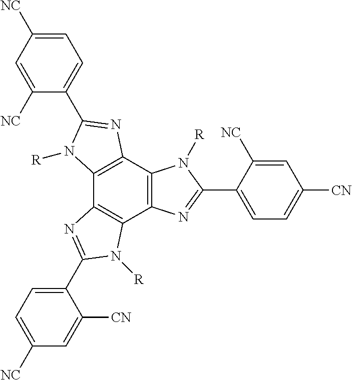

| | |
|---|---|
| GI-61 | H |
| GI-62 | 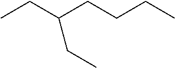 |
| GI-63 | 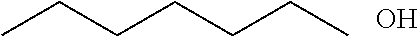 |
| GI-64 | 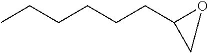 |
| GI-65 | 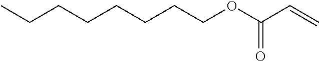 |

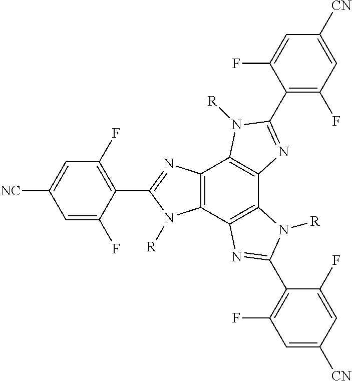

| | |
|---|---|
| GI-66 | H |
| GI-67 | 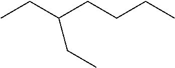 |
| GI-68 | 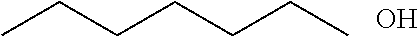 |
| GI-69 | 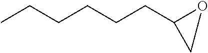 |
| GI-70 | 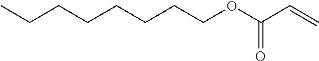 |

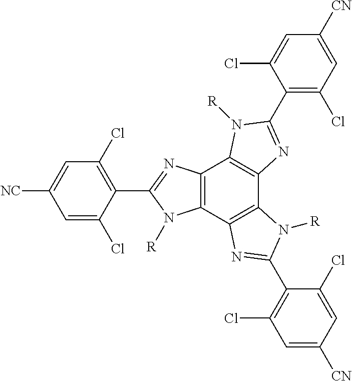

| | |
|---|---|
| GI-71 | H |
| GI-72 | 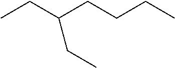 |
| GI-73 | 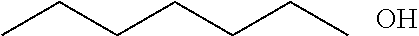 |
| GI-74 | 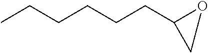 |
| GI-75 | 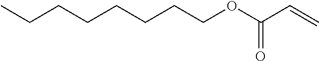 |

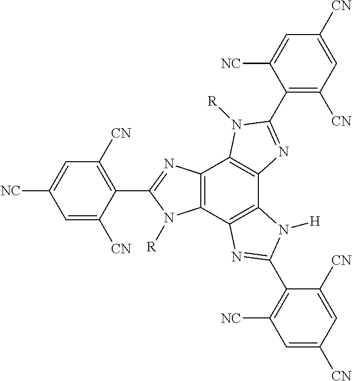

| | |
|---|---|
| GI-76 | H |
| GI-77 | 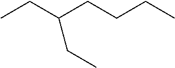 |
| GI-78 | 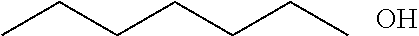 |
| GI-79 | 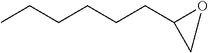 |
| GI-80 | 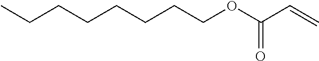 |

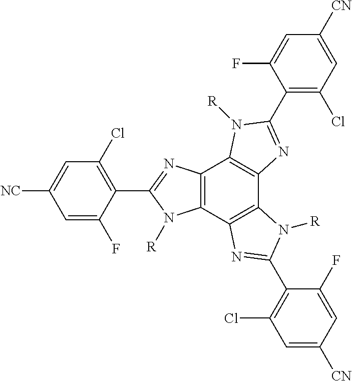

| | |
|---|---|
| GI-81 | H |
| GI-82 | 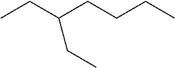 |
| GI-83 | 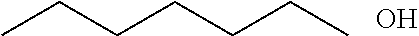 |
| GI-84 | 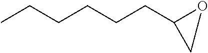 |
| GI-85 | 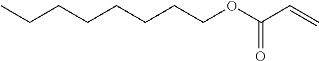 |

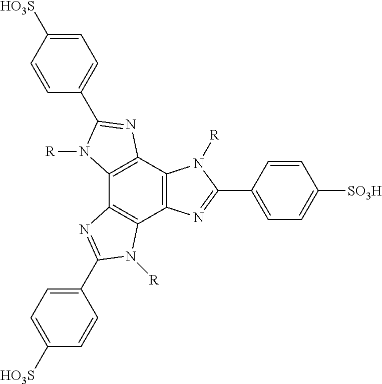

| | |
|---|---|
| GI-86 | H |
| GI-87 | 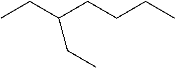 |
| GI-88 | 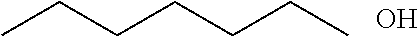 |
| GI-89 | 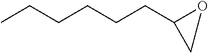 |
| GI-90 | 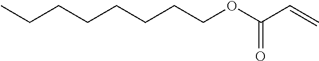 |

In the radicals show in Table A, the linkage to the core molecule is the bond shown to the left (e.g., the radical of Compound GI-87 has 8 carbons).

In a Group II subset of compounds, R¹ is aryl. Nonlimiting examples of Group II include compounds where R¹ and R² are:

TABLE B

| | |
|---|---|
| GII-01 | 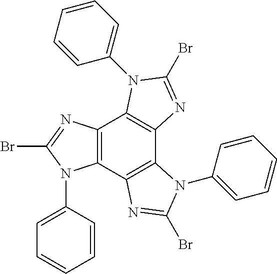 |
| GII-02 | 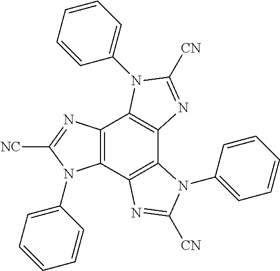 |
| GII-03 | 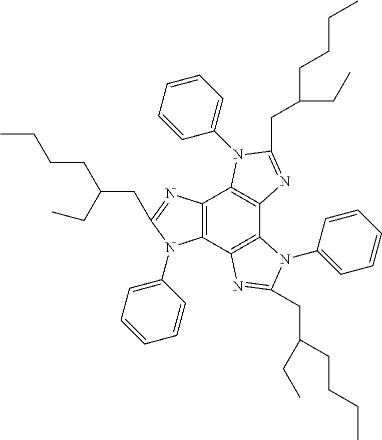 |
| GII04 | 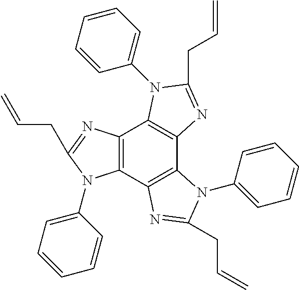 |
| GII-05 | 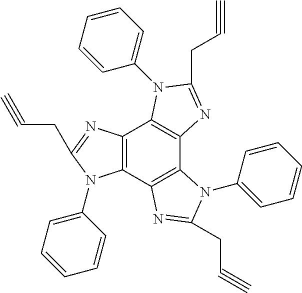 |
| GII-06 | 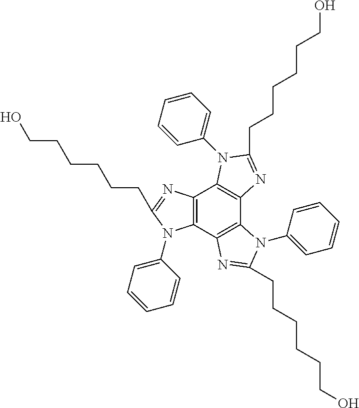 |
| GII-07 | 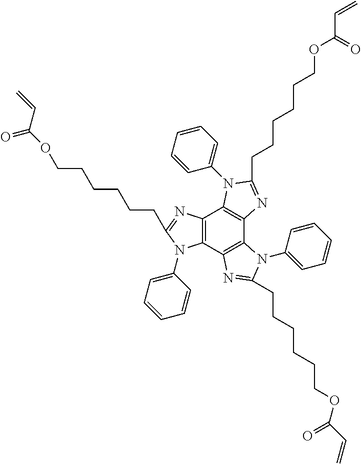 |
| GII-08 | 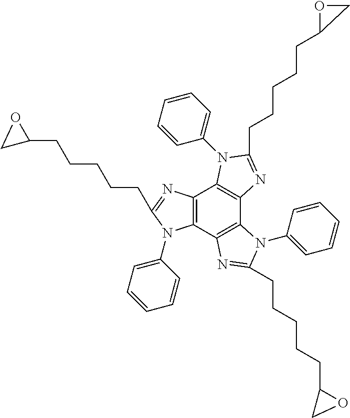 |

In a Group III subset of compounds, R¹ and R² are aryl. Nonlimiting examples of Group III include compounds where R¹ and R² are:

TABLE C

| | |
|---|---|
| GIII-01 | 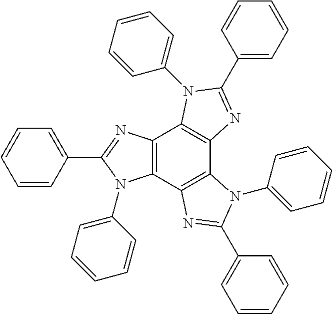 |
| GIII-02 | 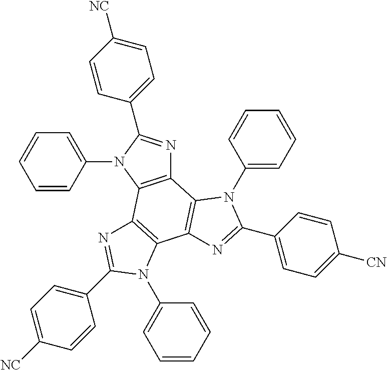 |
| GIII-03 | 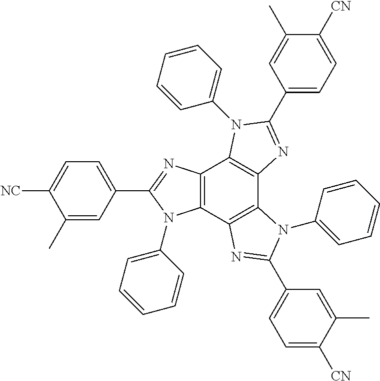 |
| GIII-04 | 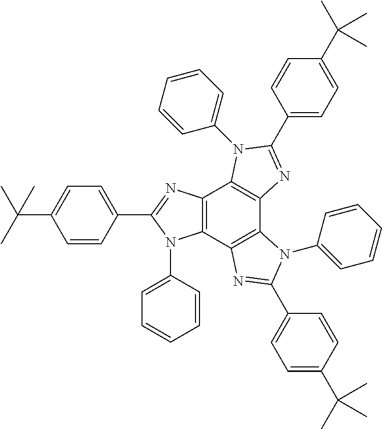 |
| GIII-05 | 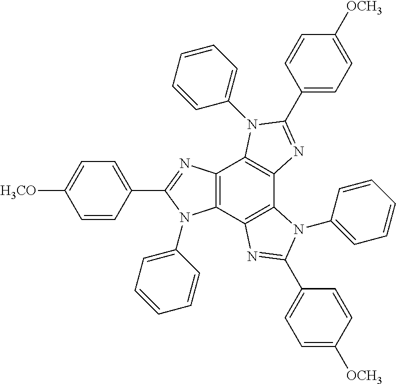 |
| GIII-06 | 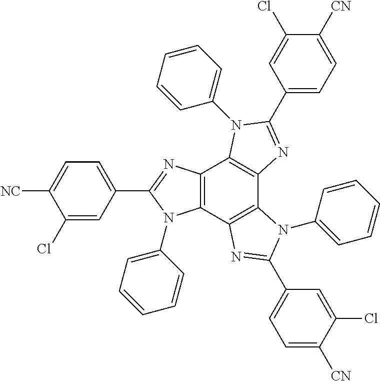 |
| GIII-07 | 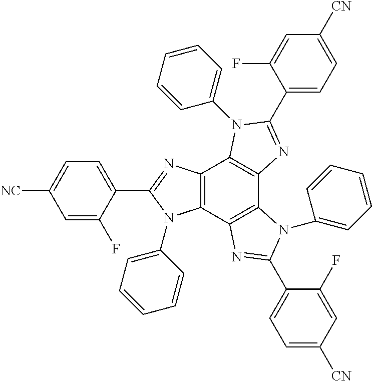 |
| GIII-08 | 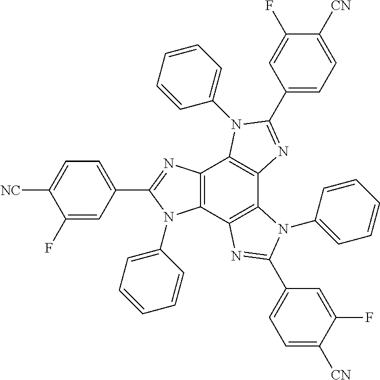 |
| GIII-09 | 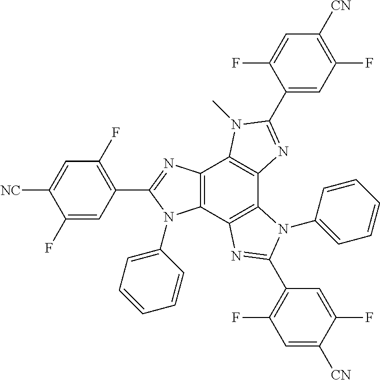 |
| GIII-10 | 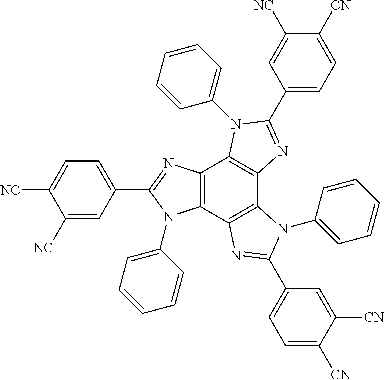 |
| GIII-11 | 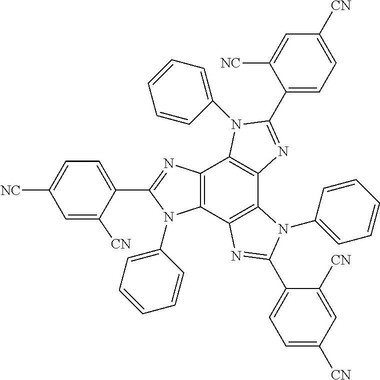 |
| GIII-12 | 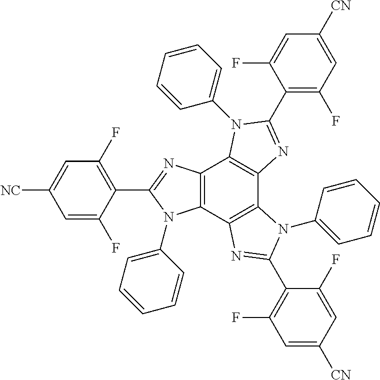 |
| GIII-13 | 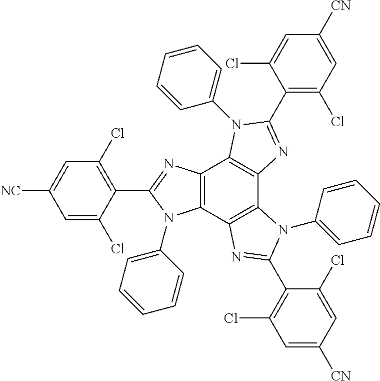 |
| GIII-14 | 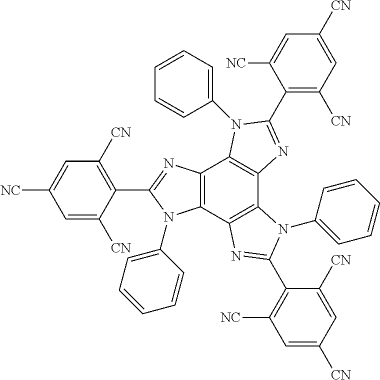 |
| GIII-15 | 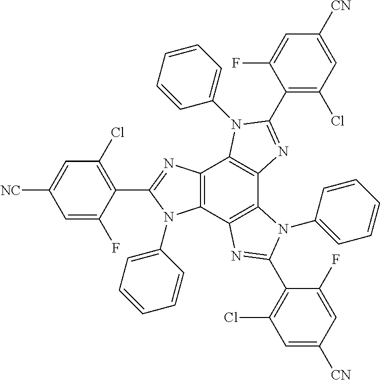 |
| GIII-16 | 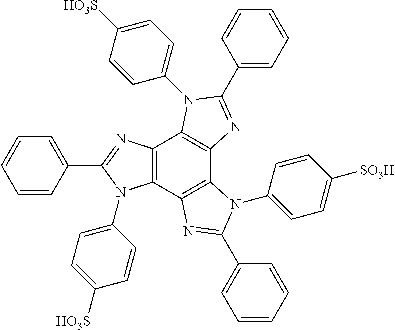 |
| GIII-17 | 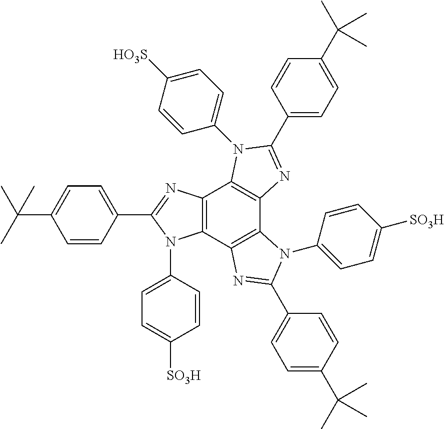 |
| GIII-18 | 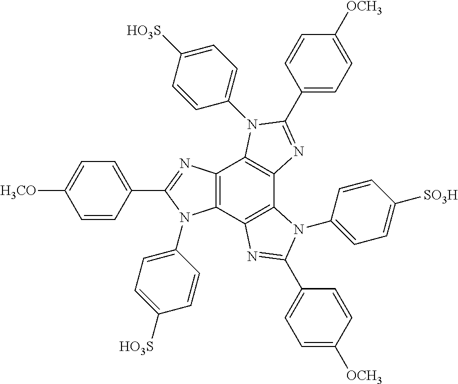 |
| GIII-19 | 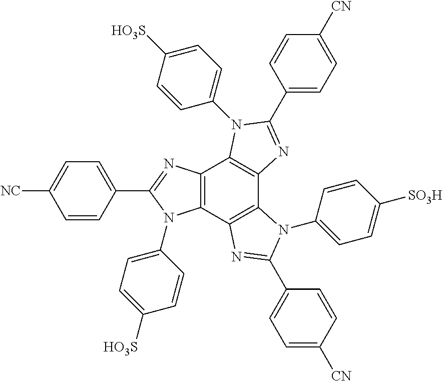 |
| GIII-20 | 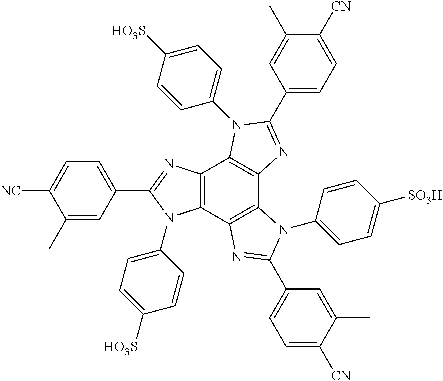 |
| GIII-21 | 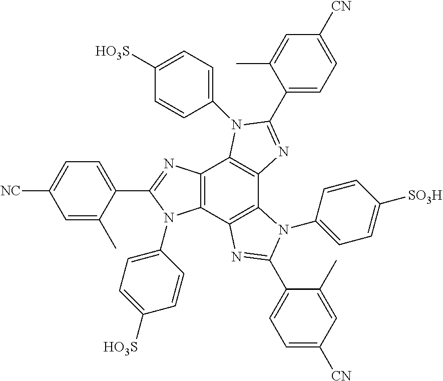 |
| GIII-22 | 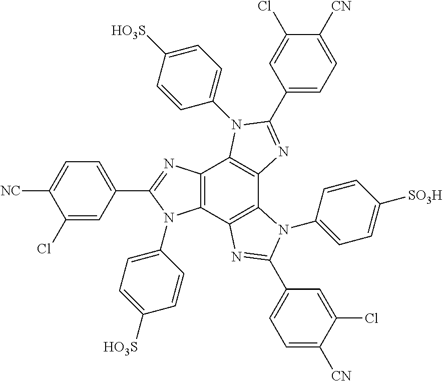 |
| GIII-23 | 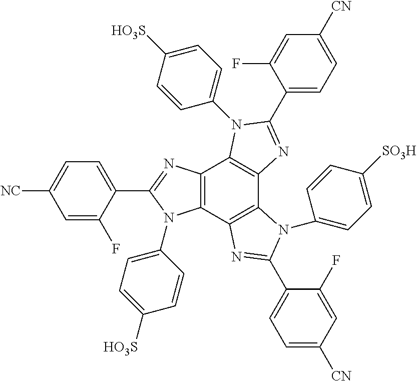 |
| GIII-24 | 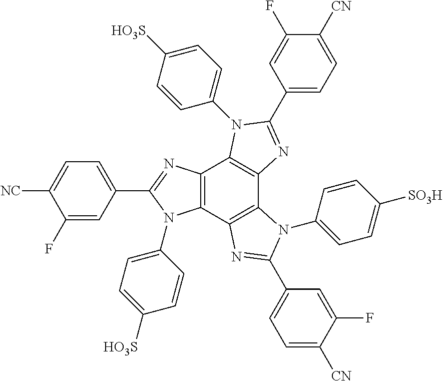 |
| GIII-25 | 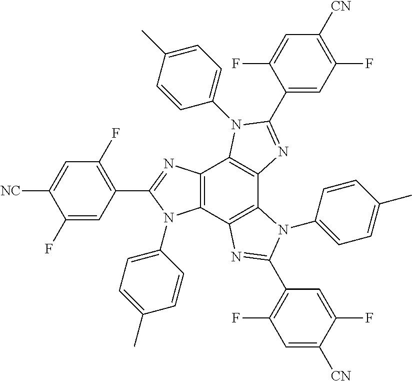 |
| GIII-26 | 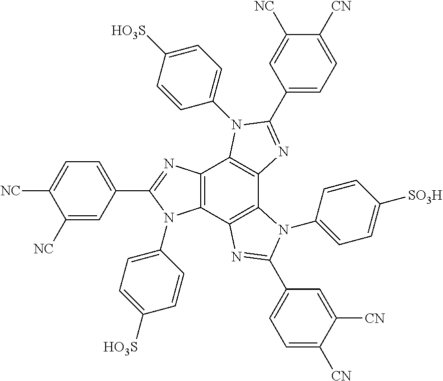 |
| GIII-27 | 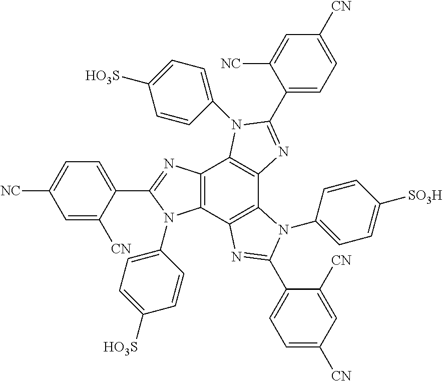 |
| GIII-28 | 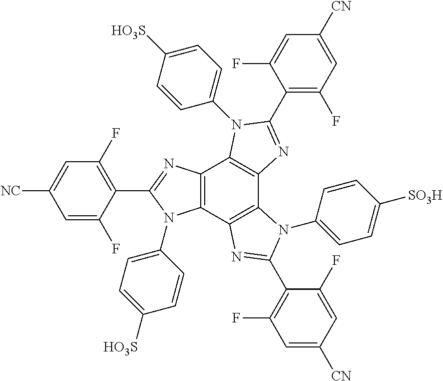 |
| GIII-29 | 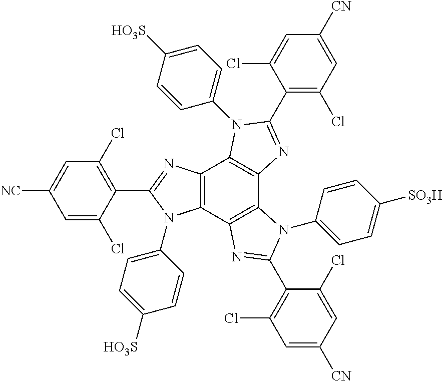 |
| GIII-30 | 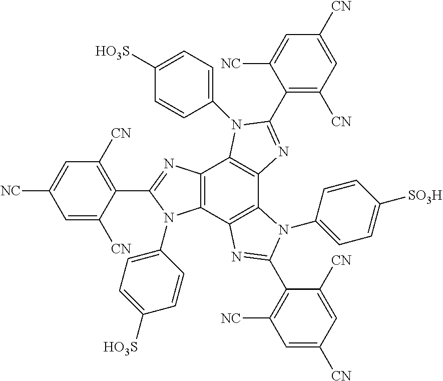 |
| GIII-31 | 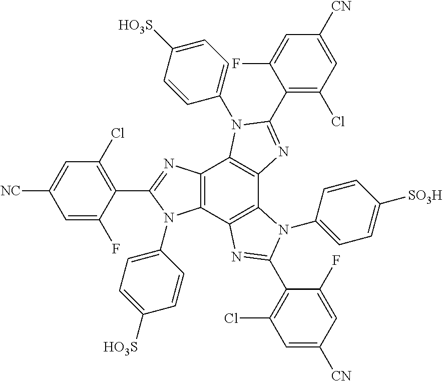 |
| GIII-32 | 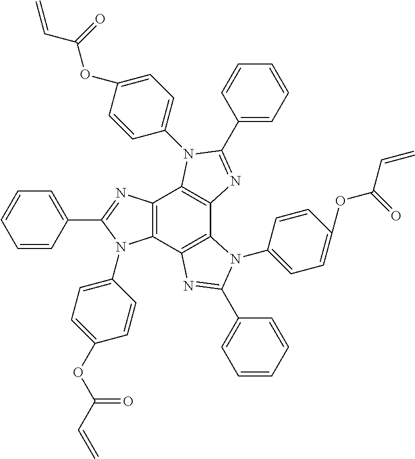 |
| GIII-33 | 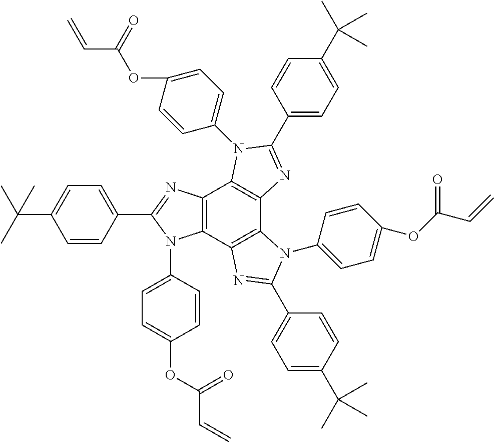 |
| GIII-34 | 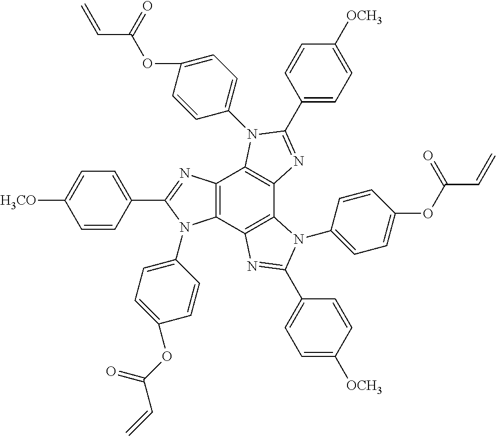 |
| GIII-35 | 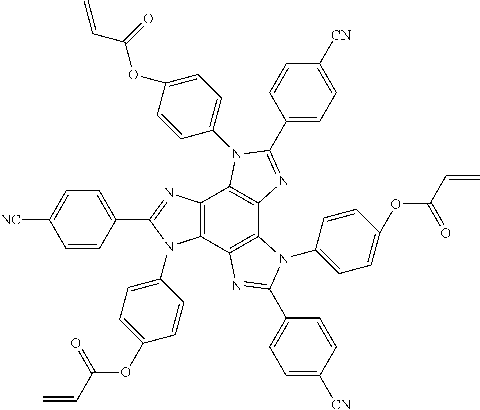 |
| GIII-36 | 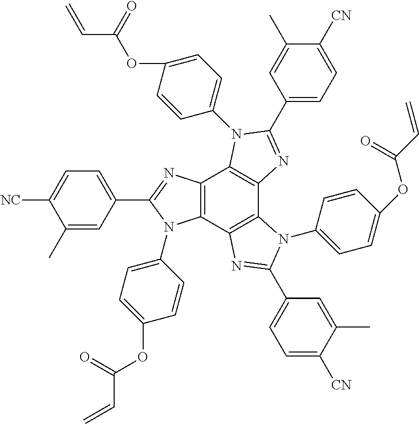 |
| GIII-37 | 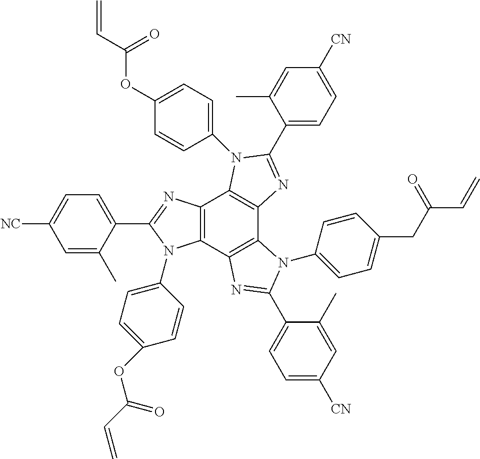 |
| GIII-38 | 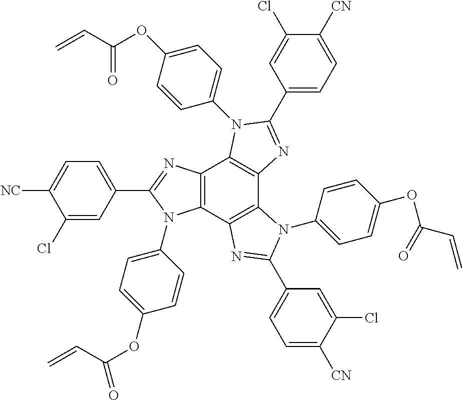 |
| GIII-39 | 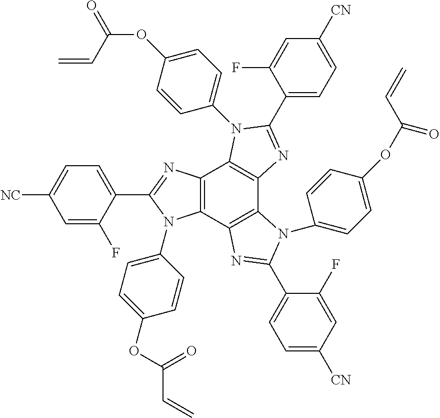 |
| GIII-40 | 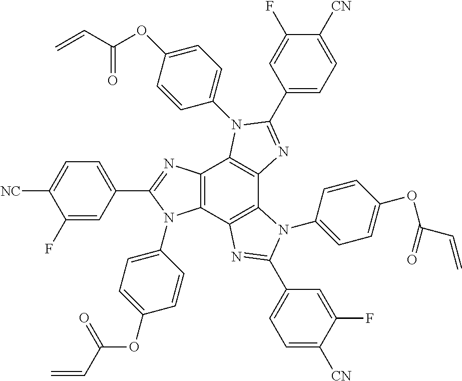 |
| GIII-41 | 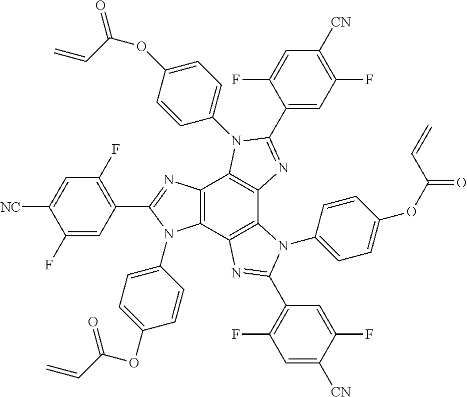 |
| GIII-42 | 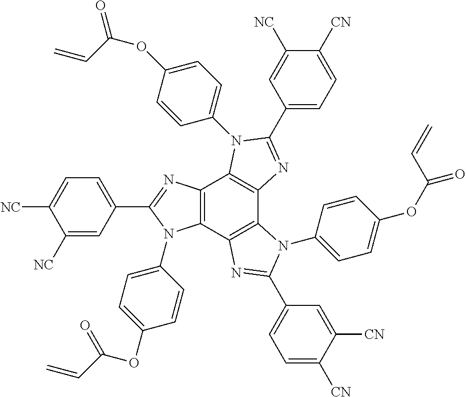 |
| GIII-43 | 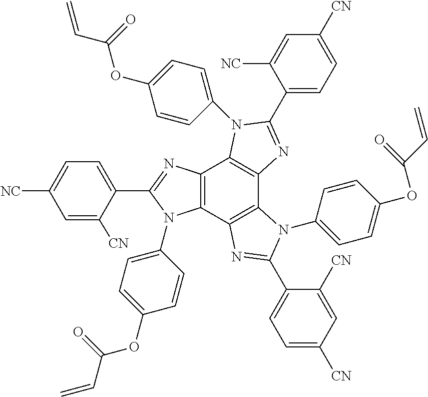 |
| GIII-44 | 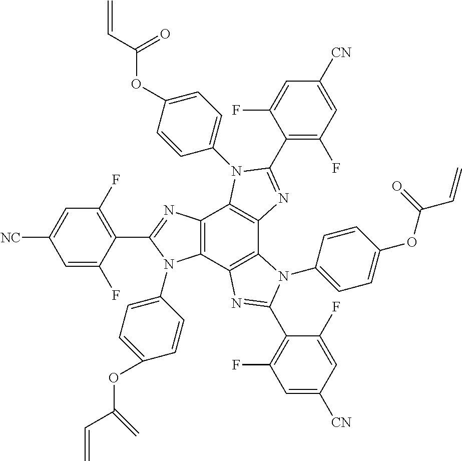 |
| GIII-45 | 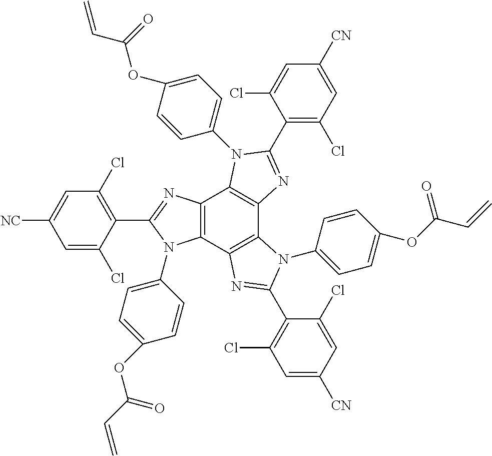 |
| GIII-46 | 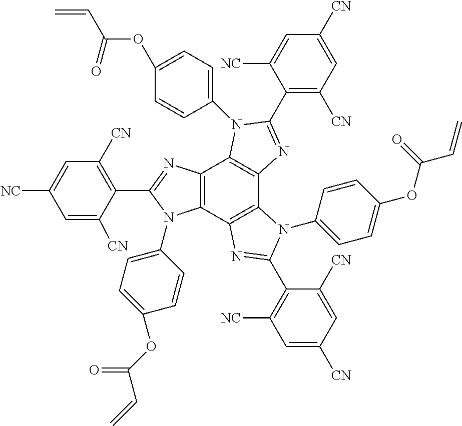 |
| GIII-47 | 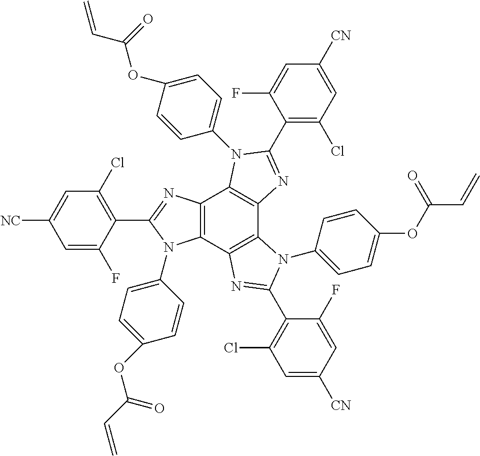 |

While the above illustrates a single species for R¹ or R², one of skill will recognize from the reaction scheme that a mixture of R groups can be obtained at the various instances of R¹ or R².

The compounds of the invention can be used for example in organic electroluminescent devices, organic solid state light devices, organic photovoltaics, and like organic opto-electron applications.

An exemplary EL, an OLED, is shown in FIG. 8. FIG. 8 shows an illustrative OLED assembly 10. The OLED assembly can include for example anode 13, which can include an ITO coating on a glass or quartz support 12. A hole injecting or transporting layer 14 can be atop of the ITO coating. A light emitting layer 15 comprising of light emitting material (comprising compound of the invention) can be disposed atop layer 14. A electron transport layer 16 can be disposed on layer 15. Finally, a cathode 17, for example in form of a thin metallic film can be atop layer 16. Light emissions are indicated with arrows.

A photovoltaic device where compounds according to the invention can be used to store light energy for use in low sunlight are described for example in WO2012045924A1 (the description therein of such a device is incorporated herein in its entirety).

Specific embodiments according to the methods of the present invention will now be described in the following examples. The examples are illustrative only, and are not intended to limit the remainder of the disclosure in any way.

All ranges recited herein include ranges therebetween, and can be inclusive or exclusive of the endpoints. Optional included ranges are from integer values therebetween (or inclusive of one original endpoint), at the order of magnitude recited or the next smaller order of magnitude. For example, if the lower range value is 0.2, optional included endpoints can be 0.3, 0.4, . . . 1.1, 1.2, and the like, as well as 1, 2, 3 and the like; if the higher range is 8, optional included endpoints can be 7, 6, and the like, as well as 7.9, 7.8, and the like. One-sided boundaries, such as 3 or more, similarly include consistent boundaries (or ranges) starting at integer values at the recited order of magnitude or one lower. For example, 3 or more includes 4 or more, or 3.1 or more.

Where a sentence states that its subject is found in embodiments, or in certain embodiments, or in the like, it is applicable to any embodiment in which the subject matter can be logically applied.

### EXAMPLE I

### Synthesis of 1,2,4,5,7,8-hexaphenyl-4,7-dihydro-1H-benzo-[1,2-d:3,4-d′:5,6-d″]triimidazole

1,3,5-triphenylamino-2,4,6-triaminobenzene (2.0 g, 5 mmol) and benzoyl chloride (2.6 g, 16 mmol) were dissolved in anhydrous N-methyl-2-pyrrolidone (30 mL). The resulting mixture was stirred at room temperature for one hour. The reaction mixture was precipitated in 500 mL of water and crude N,N′,N″-(2,4,6-tris(phenylamino)benzene-1,3,5-triyl)tris(benzamide) product as a gray/purple precipitate was isolated by filtration and allowed to air dry on the filter overnight.

The crude N,N′,N″-(2,4,6-tris(phenylamino)benzene-1,3,5-triyl)tris(benzamide) obtained above was dissolved in glacial acetic acid (75 mL) without further purification. The reaction mixture was refluxed for 16 hours and then, after cooling to room temperature, was neutralized with sodium bicarbonate and filtered to isolate the brown precipitate as a crude product. The crude product was dissolved in a minimum amount of dichloromethane and then loaded onto a silica gel column for chromatography using dichloromethane as an eluent. The product was collected as a yellow/brown fraction and the volume of the solution was reduced by evaporation. Hexane was added to the product solution with stirring and a light yellow solid precipitated from the solution. The precipitate was collected as a light yellow solid by vacuum filtration and remaining impurities were washed away with minimal cold dichloromethane to leave pure 1,2,4,5,7,8-hexaphenyl-4,7-dihydro-1H-benzo[1,2-d:3,4-d′:5,6-d″]triimidazole as a white solid (0.97 g, 1.48 mmol, 30% yield). ¹H NMR (400 MHz, CDCl₃, δ): 7.63-7.59 (m, 2H), 7.55-7.50 (m, 3H), 7.43 (m, 2H), 7.23-7.16 (m, 3H). ¹³C (CDCl₃, δ): 148.65, 138.17, 130.69, 128.99, 128.92, 128.79, 128.55, 128.32, 127.94, 127.09, 126.15.

### EXAMPLE II

### Synthesis of 2,5,8-tris(4-tert-butyl-phenyl)-1,4,7-triphenyl-4,7-dihydro-1H-benzo[1,2-d:3,4-d′:5,6-d″]triimidazole

1,3,5-triphenylamino-2,4,6-triaminobenzene (2.0 g 5 mmol) and 4-tert-butyl-benzoylchloride (3.14 g, 16 mmol) were dissolved in anhydrous N-methyl-2-pyrrolidone (30 mL). The resulting mixture was stirred at room temperature for one hour. The reaction mixture was precipitated in 500 mL of water and crude N,N′,N″-(2,4,6-tris(phenylamino)benzene-1,3,5-triyl)tris(4-tert-butyl-benzamide) product as a gray/purple precipitate was isolated by filtration and allowed to air dry on the filter overnight.

The crude N,N′,N″-(2,4,6-tris(phenylamino)benzene-1,3,5-triyl)tris(4-tert-butyl-benzamide) obtained above was dissolved in glacial acetic acid (75 mL) without further purification. The reaction mixture was refluxed for 16 hours and then, after cooling to room temperature, was neutralized with sodium bicarbonate and filtered to isolate the brown precipitate as a crude product. The brown solid was purified by silica gel column chromatography with an eluent of 20% ethyl acetate and 80% hexane. The purified product was collected as a clear solution with blue/purple photoluminescence and the solvent was removed by evaporation to yield the pure compound 2,5,8-tris(4-tert-butyl-phenyl)-1,4,7-triphenyl-4,7-dihydro-1H-benzo[1,2-d:3,4-d′:5,6-d″]triimidazole as a white solid product (1.45 g, 1.65 mmol, 33% yield). ¹H NMR (400 MHz, CDCl₃, δ): 7.66-7.60 (m, 2H), 7.58-7.51 (m, 3H), 7.35 (d, J=8.8 Hz, 2H), 7.19 (d, J=8.8 Hz, 2H), 1.25 (s, 9H). ¹³C (CDCl₃, δ): 151.29, 148.55, 138.42, 128.89, 128.46, 127.87, 127.02, 126.00, 124.92, 34.60, 31.22.

### EXAMPLE III

### Synthesis of 2,5,8-tris(4-cyanophenyl)-1,4,7-triphenyl-4,7-dihydro-1H-benzo[1,2-d:3,4-d′:5,6-d″]triimidazole

1,3,5-triphenylamino-2,4,6-triaminobenzene (2.0 g, 5 mmol) and 4-cyanobenzoyl chloride (2.6 g, 16 mmol) were dissolved in anhydrous N-methyl-2-pyrrolidone (30 mL). The resulting mixture was stirred at room temperature for one hour. The reaction mixture was precipitated in 500 mL of water and crude N,N′,N″-(2,4,6-tris(phenylamino)benzene-1,3,5-triyl)tris(4-cyanobenzamide) product as a gray/brown precipitate was isolated by filtration and allowed to air dry on the filter overnight.

The crude N,N′,N″-(2,4,6-tris(phenylamino)benzene-1,3,5-triyl)tris(4-cyanobenzamide) obtained above was dissolved in glacial acetic acid (75 mL) without further purification. The reaction mixture was refluxed for 16 hours and then, after cooling to room temperature, was neutralized with sodium bicarbonate and filtered to isolate the yellow/brown precipitate as a crude product. The crude product was dissolved in a minimum amount of dichloromethane and then loaded onto a silica gel column for chromatography using dichloromethane as an eluent. The purified product was separated from a brown impurity and collected as a clear solution with bright blue photoluminescence. The solvent was removed by evaporation and the pure compound of 2,5,8-tris(4-cyanophenyl)-1,4,7-triphenyl-4,7-dihydro-1H-benzo[1,2-d:3,4-d′:5,6-d″]triimidazole was obtained as a white solid (1.13 g, 1.55 mmol, 31% yield). ¹H NMR (400 MHz, CDCl₃, δ): 7.62-7.54 (m, 3H), 7.52-7.44 (m, 2H). ¹³C (CDCl₃, δ): 146.85, 137.43, 134.51, 131.86, 129.47, 128.96, 128.42, 127.47, 127.13, 118.57, 111.85.

### EXAMPLE IV

### Synthesis of 2,5,8-tris(4-methoxyphenyl)-1,4,7-triphenyl-4,7-dihydro-1H-benzo[1,2-d:3,4-d′:5,6-d″]triimidazole

1,3,5-triphenylamino-2,4,6-triaminobenzene (2.0 g, 5 mmol) and 4-methoxy benzoyl chloride (2.72 g, 16 mmol) were dissolved in anhydrous N-methyl-2-pyrrolidone (30 mL). The resulting mixture was stirred at room temperature for one hour. The reaction mixture was precipitated in 500 mL of water and crude N,N′,N″-(2,4,6-tris(phenylamino)benzene-1,3,5-triyl)tris(4-methoxybenzamide) product as a gray/purple precipitate was isolated by filtration and allowed to air dry on the filter overnight.

The crude N,N′,N″-(2,4,6-tris(phenylamino)benzene-1,3,5-triyl)tris(4-methoxybenzamide) was dissolved in glacial acetic acid (75 mL) without further purification. The reaction mixture was refluxed for 16 hours and then, after cooling to room temperature, was neutralized with sodium bicarbonate and filtered to isolate the brown precipitate as a crude product. The brown solid was dissolved in dichloromethane and filtered through a plug of silica gel to remove the brown impurity. The resulting light yellow solution was dried by evaporation and the resulting yellow solid was purified using silica gel column chromatography with an eluent of 20% ethyl acetate, 80% hexane. The product was collected as a clear solution with purple photoluminescence. The solvent was removed by evaporation to leave the pure compound 2,5,8-tris(4-methoxyphenyl)-1,4,7-triphenyl-4,7-dihydro-1H-benzo[1,2-d:3,4-d′:5,6-d″]triimidazole as a white solid (1.15 g, 1.55 mmol, 31% yield). ¹H NMR (400 MHz, CDCl₃, δ): 7.61-7.58 (m, 2H), 7.57-7.50 (m, 3H), 7.38-7.33 (m, 2H), 6.73-6.69 (m, 2H), 3.75 (s, 3H).

### EXAMPLE 5

### Synthesis of 2,5,8-tris(3-fluoro-4-cyanophenyl)-1,4,7-triphenyl-4,7-dihydro-1H-benzo[1,2-d:3,4-d′:5,6-d″]triimidazole

1,3,5-triphenylamino-2,4,6-triaminobenzene (2.0 g, 5 mmol) and 3-fluoro-4-cyanobenzoyl chloride (2.94 g, 16 mmol) were dissolved in anhydrous N-methyl-2-pyrrolidone (30 mL). The resulting mixture was stirred at room temperature for one hour. The reaction mixture was precipitated in 500 mL of water and crude N,N′,N″-(2,4,6-tris(phenylamino)benzene-1,3,5-triyl)tris(3-fluoro-4-cyanobenzamide) product as a gray/brown precipitate was isolated by filtration and allowed to air dry on the filter overnight.

The crude N,N′,N″-(2,4,6-tris(phenylamino)benzene-1,3,5-triyl)tris(3-fluoro-4-cyanobenzamide) was dissolved in glacial acetic acid (75 mL) without further purification. The reaction mixture was refluxed for 16 hours and then, after cooling to room temperature, was neutralized with sodium bicarbonate and filtered to isolate the yellow/brown precipitate as a crude product. The crude product was dissolved in a minimum amount of dichloromethane and then loaded onto a silica gel column for chromatography using dichloromethane as an eluent. The purified product was separated from a brown impurity and collected as a clear solution with bright blue photoluminescence. The solvent was removed by evaporation and the pure compound of 2,5,8-tris(3-fluoro-4-cyanophenyl)-1,4,7-triphenyl-4,7-dihydro-1H-benzo[1,2-d:3,4-d′:5,6-d″]triimidazole was obtained as a white solid (1.14 g, 1.45 mmol, 29% yield). ¹H NMR (400 MHz, CDCl₃, δ): 7.68-7.62 (m, 3H), 7.57-7.54 (m, 2H), 7.41 (dd, J=8.4 Hz, J′=6.6 Hz, 1H), 7.29 (dd, J=10.2 Hz, J′=1.2 Hz, 1H), 7.21 (dd, J=7.8 Hz, J′=1.2 Hz, 1H).

### EXAMPLE 6

An organic EL device is illustrated in the inset of FIG. 6. In such a device compound GIII-02 was doped with an example phosphorescent emitter at 7% atomic weight, namely IrPQ, in the emission layer. The EL device as shown in the inset of FIG. 10 was fabricated as follows: (a) An indium-tin-oxide (ITO) coated glass substrate was sequentially ultrasonicated in a commercial detergent, rinsed in deionized water, and exposed to RF-plasma in an oxygen atmosphere; (b) Onto the ITO layer was deposited a hole transport layer of N,N′-bis-(1-naphthyl)-N,N′-diphenylbenzidine (NPB) with a thickness of 750 Å, by evaporation from a tantalum boat; (c) A emission layer of the invented compound, GIII-02 was co-deposited with an example phosphorescent emitter, IrPQ, at 7% by atomic weight at a thickness of 200 Å; (d) An electron-injection layer, TPBI, was then deposited onto the emission layer at a thickness of 300 Å; (e) On top of the electron injection layer 318 was deposited a cathode layer 306 with a thickness of 2000 Å formed of a 10:1 atomic ratio of magnesium (Mg) and silver (Ag). The device was then hermetically packaged in a dry glove box for protection against the ambient environment.

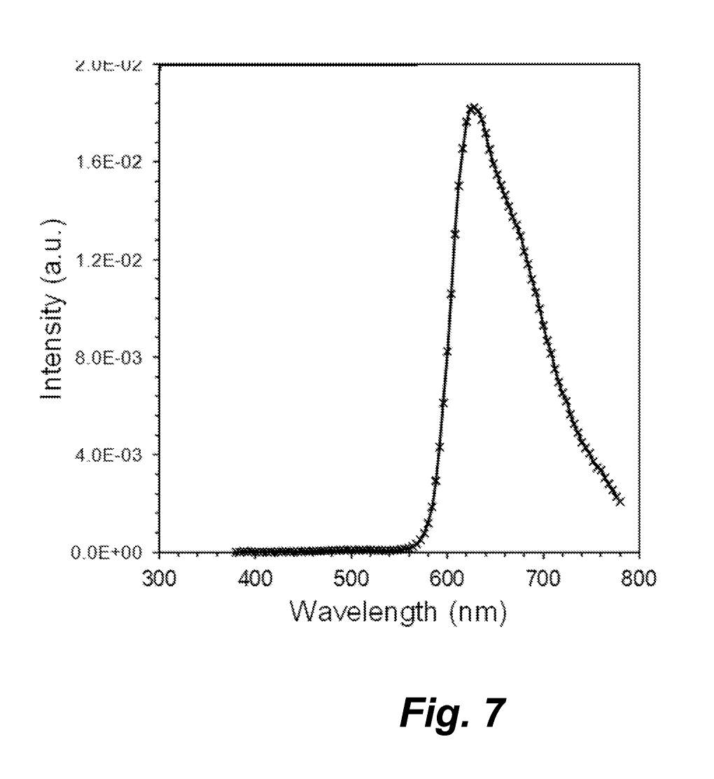

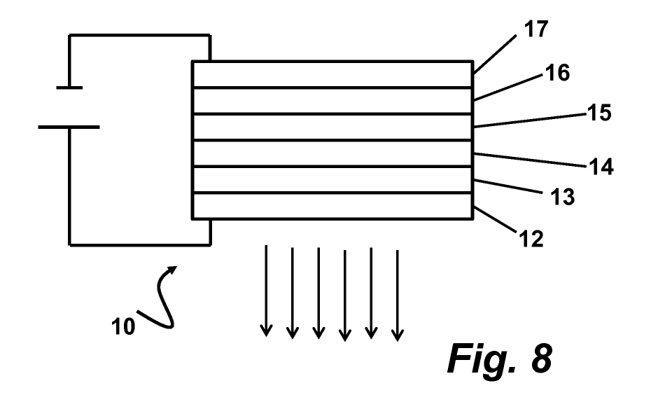

FIG. 6 shows the efficacy (cd/A as a function of current density) produced with the device. FIG. 7 shows the emission spectrum from the compound. The results demonstrate the invented material can be used in a typical OLED application. The emission in FIG. 7 demonstrates the electron and holes transport in the invented compound GIII-02 forming an exciton that energy transfers to the example phosphorescent emitter, IrPQ with the characteristic red-spectrum. As shown above, utility in an organic EL is readily confirmed for a given compound of the invention.

This invention described herein is of a aromatic benzoimidizole organic materials, methods of forming the same, and methods and devices utilizing the same. Although some embodiments have been discussed above, other implementations and applications are also within the scope of the following claims. Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the following claims.

Publications and references, including but not limited to patents and patent applications, cited in this specification are herein incorporated by reference in their entirety in the entire portion cited as if each individual publication or reference were specifically and individually indicated to be incorporated by reference herein as being fully set forth. Any patent application to which this application claims priority is also incorporated by reference herein in the manner described above for publications and references.

1. (canceled)

2. An aromatic benzotriimizole compound of Formula I: 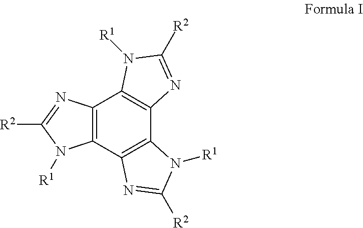
wherein,
R¹ is independently H, alkyl or aryl, wherein alkyl and the substitutions of aryl can include alkyl substituted with a reactive moiety; and
R² is independently, chloro, fluoro, bromo or cyano, or wherein R² is aryl.

3. (canceled)

4. The compound of claim 2, wherein
R² is aryl.

5. The compound of claim 2, wherein R¹ is selected from the group consisting of

| GI-01 | H |
|---|---|
| GI-02 | 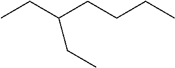 |
| GI-03 | 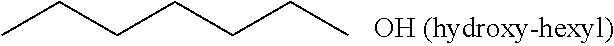 |
| GI-04 | 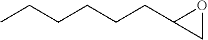 |
| GI-05 | 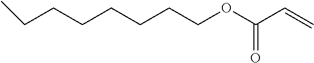 |
| 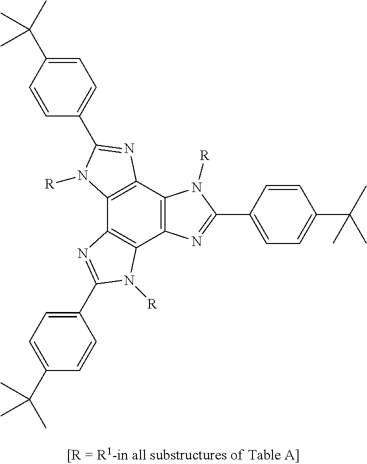 | |
| GI-06 | H |
| GI-07 | 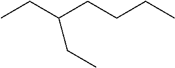 |
| GI-08 | 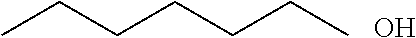 |
| GI-09 | 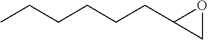 |
| GI-10 | 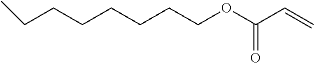 |
| 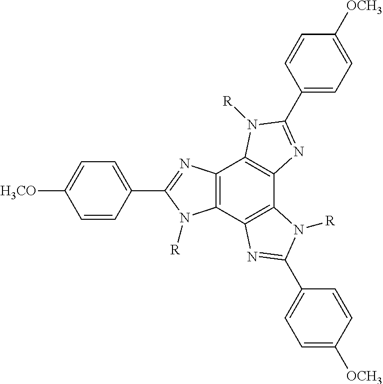 | |
| GI-11 | H |
| GI-12 | 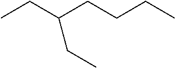 |
| GI-13 | 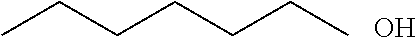 |
| GI-14 | 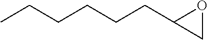 |
| GI-15 | 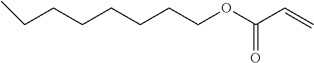 |
| 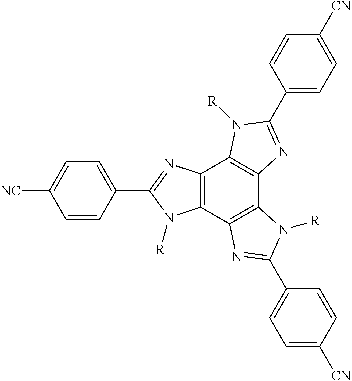 | |
| GI-16 | H |
| GI-17 | 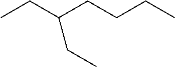 |
| GI-18 | 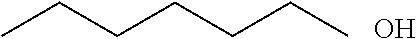 |
| GI-19 | 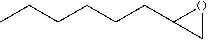 |
| GI-20 | 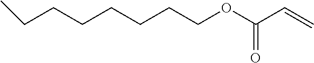 |
| 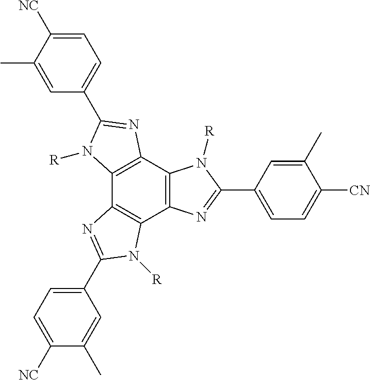 | |
| GI-21 | H |
| GI-22 | 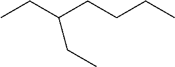 |
| GI-23 | 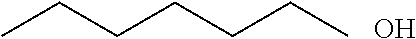 |
| GI-24 | 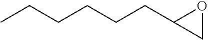 |
| GI-25 | 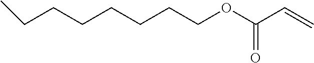 |
| 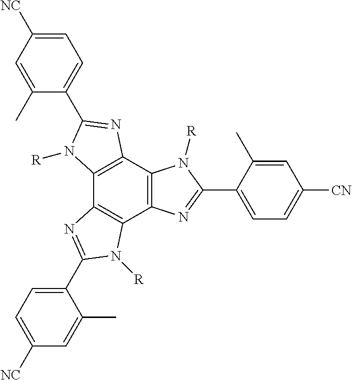 | |
| GI-26 | H |
| GI-27 | 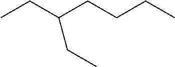 |
| GI-28 | 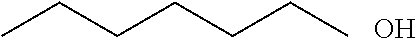 |
| GI-29 | 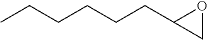 |
| GI-30 | 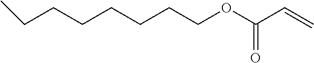 |
| 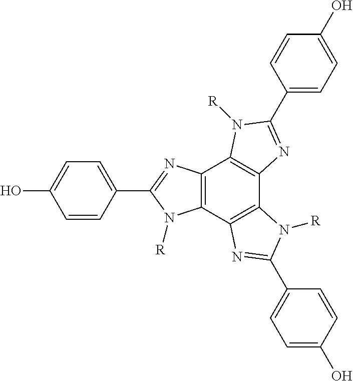 | |
| GI-31 | H |
| GI-32 | 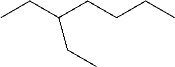 |
| GI-33 | 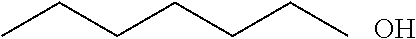 |
| GI-34 | 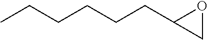 |
| GI-35 | 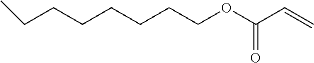 |
| 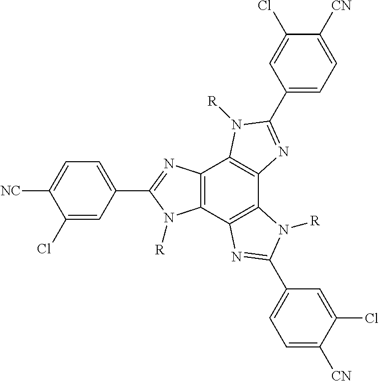 | |
| GI-36 | H |
| GI-37 | 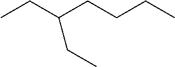 |
| GI-38 | 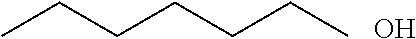 |
| GI-39 | 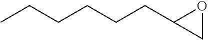 |
| GI-40 | 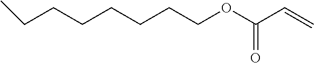 |
| 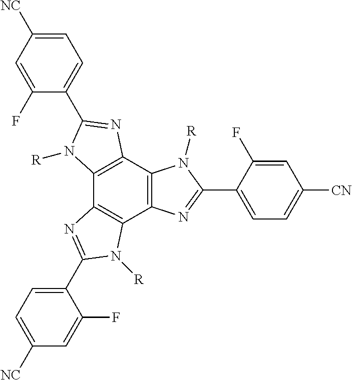 | |
| GI-41 | H |
| GI-42 | 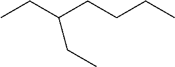 |
| GI-43 | 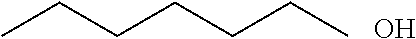 |
| GI-44 | 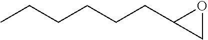 |
| GI-45 | 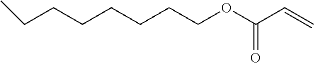 |
| 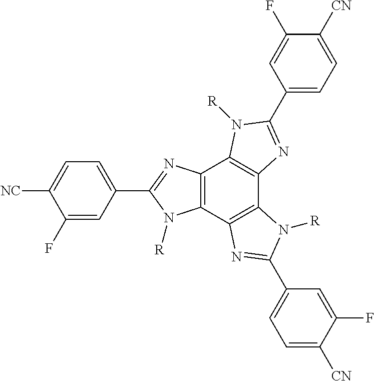 | |
| GI-46 | H |
| GI-47 | 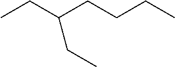 |
| GI-48 | 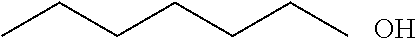 |
| GI-49 | 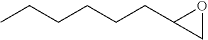 |
| GI-50 | 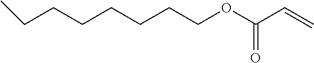 |
| 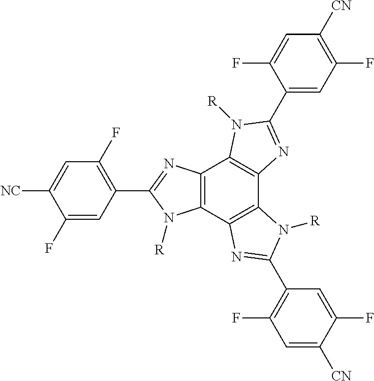 | |
| GI-51 | H |
| GI-52 | 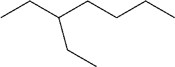 |
| GI-53 | 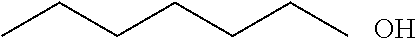 |
| GI-54 | 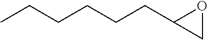 |
| GI-55 | 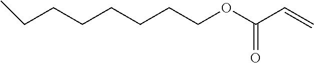 |
| 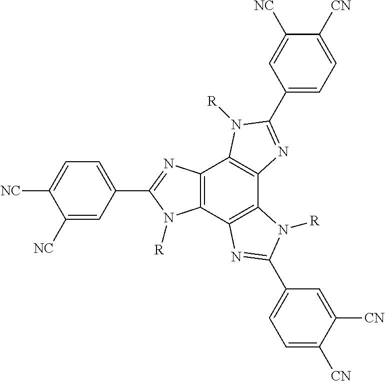 | |
| GI-56 | H |
| GI-57 | 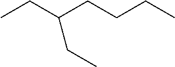 |
| GI-58 | 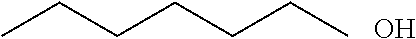 |
| GI-59 | 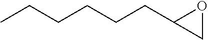 |
| GI-60 | 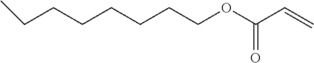 |
| 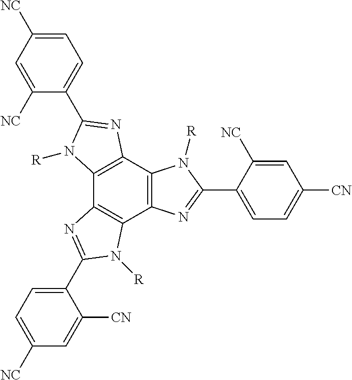 | |
| GI-61 | H |
| GI-62 | 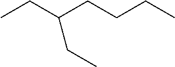 |
| GI-63 | 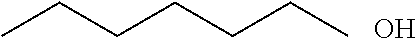 |
| GI-64 | 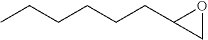 |
| GI-65 | 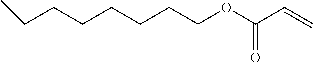 |
| 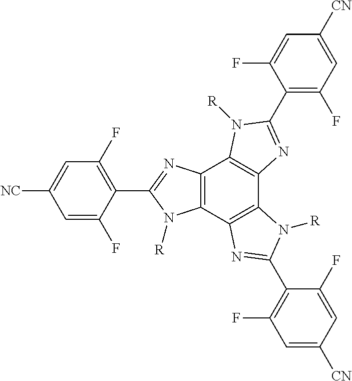 | |
| GI-66 | H |
| GI-67 | 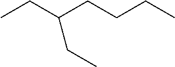 |
| GI-68 | 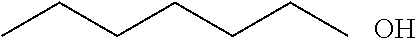 |
| GI-69 | 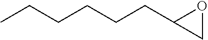 |
| GI-70 | 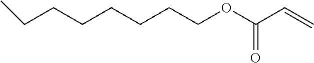 |
| 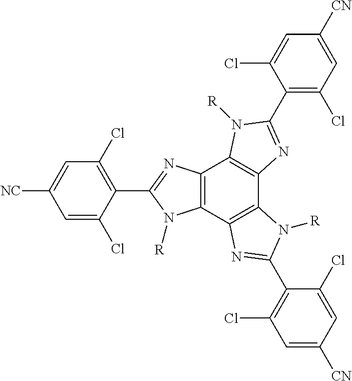 | |
| GI-71 | H |
| GI-72 | 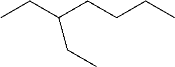 |
| GI-73 | 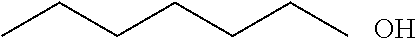 |
| GI-74 | 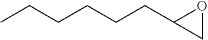 |
| GI-75 | 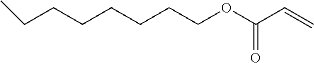 |
| 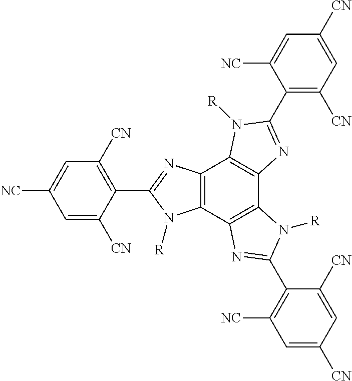 | |
| GI-76 | H |
| GI-77 | 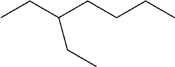 |
| GI-78 | 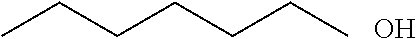 |
| GI-79 | 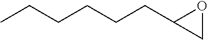 |
| GI-80 | 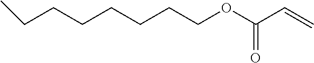 |
| 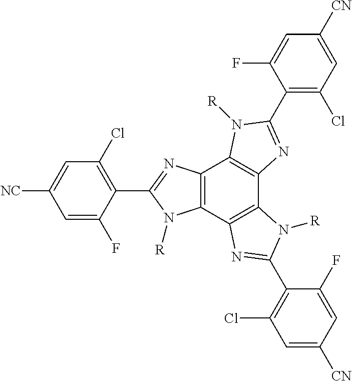 | |
| GI-81 | H |
| GI-82 | 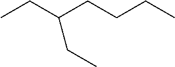 |
| GI-83 | 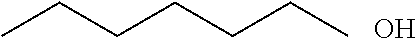 |
| GI-84 | 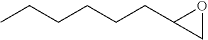 |
| GI-85 | 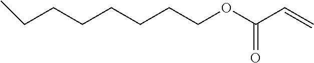 |
| 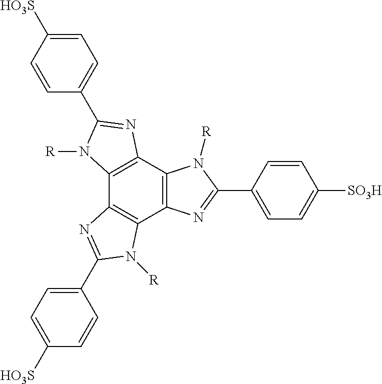 | |
| GI-86 | H |
| GI-87 | 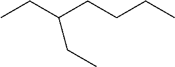 |
| GI-88 | 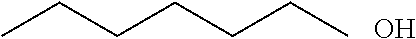 |
| GI-89 | 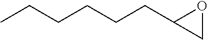 |
| GI-90 | 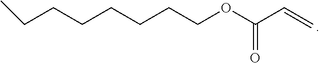 |

6. The compound of claim 2, wherein the compound is 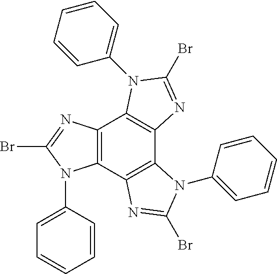 [SMILES: Brc1nc2c(c3nc(Br)n(-c4ccccc4)c3c3nc(Br)n(-c4ccccc4)c23)n1-c1ccccc1]
Compound GII-01.

7. The compound of claim 2, wherein the compound is

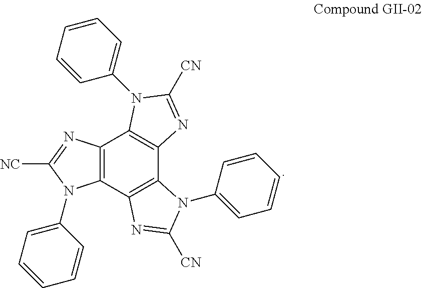 [SMILES: N#Cc1nc2c(c3nc(C#N)n(-c4ccccc4)c3c3nc(C#N)n(-c4ccccc4)c23)n1-c1ccccc1]

8. The compound of claim 2, wherein the compound is a 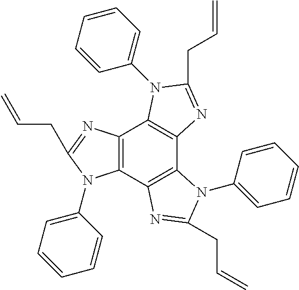 [SMILES: C=CCc1nc2c(c3nc(CC=C)n(-c4ccccc4)c3c3nc(CC=C)n(-c4ccccc4)c23)n1-c1ccccc1]
Compound GII-04.

9. The compound of claim 2, wherein the compound is a 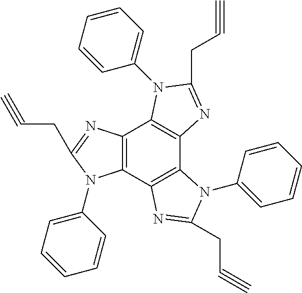 [SMILES: C#CCc1nc2c(c3nc(CC#C)n(-c4ccccc4)c3c3nc(CC#C)n(-c4ccccc4)c23)n1-c1ccccc1]
Compound GII-05.

10. The compound of claim 2, wherein the compound is 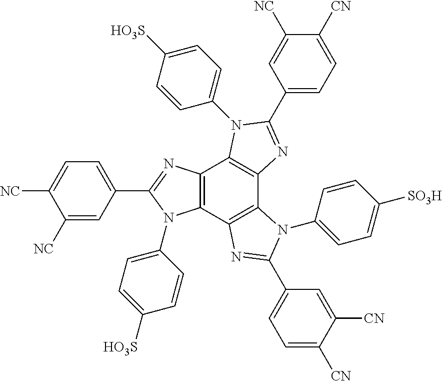 [SMILES: N#Cc1ccc(-c2nc3c(c4nc(-c5ccc(C#N)c(C#N)c5)n(-c5ccc(S(=O)(=O)O)cc5)c4c4nc(-c5ccc(C#N)c(C#N)c5)n(-c5ccc(S(=O)(=O)O)cc5)c34)n2-c2ccc(S(=O)(=O)O)cc2)cc1C#N]
Compound GIII-26.

11. The compound of claim 2, wherein the 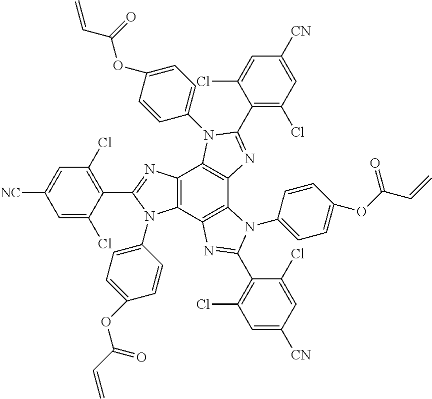 [SMILES: C=CC(=O)Oc1ccc(-n2c(-c3c(Cl)cc(C#N)cc3Cl)nc3c2c2nc(-c4c(Cl)cc(C#N)cc4Cl)n(-c4ccc(OC(=O)C=C)cc4)c2c2nc(-c4c(Cl)cc(C#N)cc4Cl)n(-c4ccc(OC(=O)C=C)cc4)c32)cc1]
compound is Compound GIII-45.

12. The compound of claim 2, wherein the 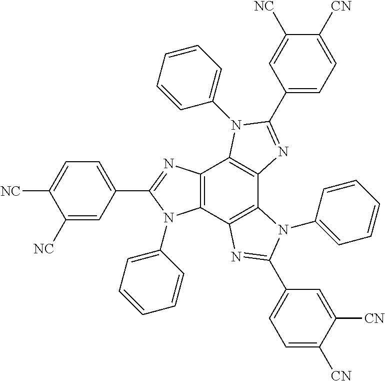 [SMILES: N#Cc1ccc(-c2nc3c(c4nc(-c5ccc(C#N)c(C#N)c5)n(-c5ccccc5)c4c4nc(-c5ccc(C#N)c(C#N)c5)n(-c5ccccc5)c34)n2-c2ccccc2)cc1C#N]
compound is Compound GIII-10.

13. The compound of claim 2, wherein the 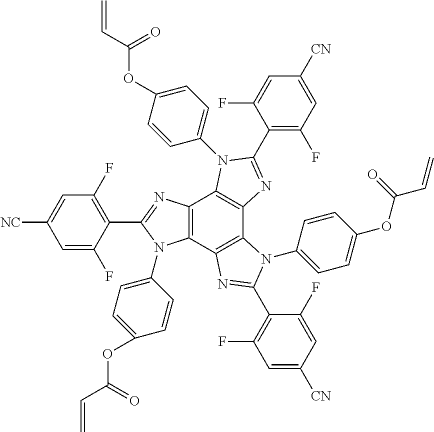 [SMILES: C=CC(=O)Oc1ccc(-n2c(-c3c(F)cc(C#N)cc3F)nc3c2c2nc(-c4c(F)cc(C#N)cc4F)n(-c4ccc(OC(=O)C=C)cc4)c2c2nc(-c4c(F)cc(C#N)cc4F)n(-c4ccc(OC(=O)C=C)cc4)c32)cc1]
compound is Compound GIII-44.

14. The compound of claim 2, wherein the compound is 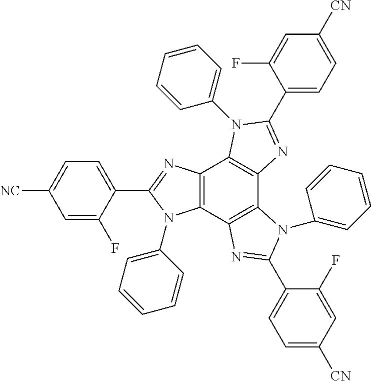 [SMILES: N#Cc1ccc(-c2nc3c(c4nc(-c5ccc(C#N)cc5F)n(-c5ccccc5)c4c4nc(-c5ccc(C#N)cc5F)n(-c5ccccc5)c34)n2-c2ccccc2)c(F)c1]
Compound GIII-07.

15. The compound of claim 2, wherein the compound is 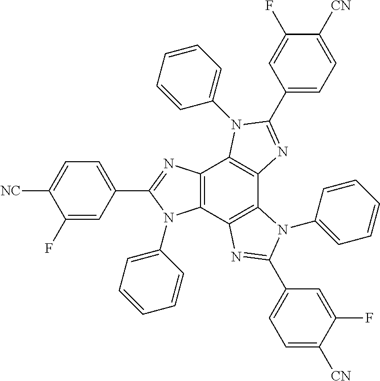 [SMILES: N#Cc1ccc(-c2nc3c(c4nc(-c5ccc(C#N)c(F)c5)n(-c5ccccc5)c4c4nc(-c5ccc(C#N)c(F)c5)n(-c5ccccc5)c34)n2-c2ccccc2)cc1F]
Compound GIII-08.

16. An EL device comprising one or more aromatic benzotriimizole compounds of claim 2 as light emitting compounds.

17. A photovoltaic device comprising one or more aromatic benzotriimizole compounds of claim 2 disposed therein to provide light emissions during low light periods that are converted to electrical energy.
